(19) United States
(12) Patent Application Publication
(10) Pub. No.: US 2012/0203147 A1
(43) Pub. Date: Aug. 9, 2012

(54) VASODILATOR-ENHANCED CARDIOPULMONARY RESUSCITATION

(75) Inventors: Keith Lurie, Minneapolis, MN (US); Demetris Yannopoulos, Saint Paul, MN (US)

(73) Assignee: ResQSystems, Inc., Roseville, MN (US)

(21) Appl. No.: 13/175,670

(22) Filed: Jul. 1, 2011

Related U.S. Application Data
(60) Provisional application No. 61/485,944, filed on May 13, 2011.
(60) Provisional application No. 61/361,208, filed on Jul. 2, 2010.

(51) Int. Cl. A61H 31/00; A61K 33/26; A61K 31/21; A61K 31/343; A61P 9/04; A61K 31/4985; A61K 31/53; A61K 31/7076; A61K 31/522; G09B 23/28; A61K 31/519

(52) U.S. Cl. 601/41; 434/265; 424/646; 514/509; 514/470; 514/252.16; 514/250; 514/243; 514/46; 514/263.34

(57) ABSTRACT

A method for increasing blood flow to vital organs during cardiopulmonary resuscitation of a person experiencing a cardiac arrest may include performing standard or active compression decompression cardiopulmonary resuscitation on a person to create artificial circulation by repetitively compressing the person's chest such that the person's chest is subject to a compression phase and a relaxation or decompression phase. The method may also include administering one or more vasodilator drugs to the person to improve the artificial circulation created by the cardiopulmonary resuscitation. The method may also include binding at least a portion of the person's abdomen, either manually or with an abdominal compression device. Performing cardiopulmonary resuscitation on a person may include ventilating the person with either an impedance threshold device or a intrathoracic pressure regulator.

### CROSS REFERENCES TO RELATED APPLICATIONS

This application is a non-provisional application and claims priority to U.S. Provisional Application No. 61/485,944, filed May 13, 2011 and to U.S. Provisional Application No. 61/361,208, filed Jul. 2, 2010, the disclosure of which is herein incorporated by reference.

### BACKGROUND OF THE INVENTION

Embodiments of the present invention relate generally to the field of cardiopulmonary resuscitation (CPR), and in particular, methods and systems for increasing for increasing the effectiveness of CPR by increasing blood perfusion to vital organs, including the heart and brain, during cardiac arrest or other heart failure.

CPR success rates have remained low over the past 50 years, with only minimal improvement in neurological intact survival rates. Even when under the care of the most experienced emergency medical service providers, blood flow generated by manual chest compression based CPR is at best less than 20% of normal levels. Additionally, because the percentage of cardiac arrest patients that present with asystole or pulseless electrical activity conditions has drastically increased to three-out-of-four in recent years, longer durations of this less-than-optimal form of CPR is being administered to patients more often.

A method of CPR, possibly including additional devices and drugs, which would significantly increase blood flow, and predominantly distribute it to vital organs such as the brain and heart, could have significant impact on resuscitation survival rates by maintaining the viability of those organs for longer periods of resuscitation.

### BRIEF DESCRIPTION OF THE INVENTION

In one embodiment, a method for increasing blood flow to vital organs during CPR of a person experiencing cardiac arrest is provided. The method proceeds by performing CPR on a person to create artificial circulation by repetitively compressing the person's chest such that the person's chest is subject to a compression phase and a relaxation or decompression phase. The method may also include administering one or more vasodilator drugs to the person to improve the artificial circulation created by the CPR. One such vasodilatory drug is sodium nitroprusside. By administering SNP, the person's blood vessels are dilated, thereby enhancing microcirculation. Nitric oxide (NO), that is released by SNP, plays an important role in regulating blood flow the heart and brain tissues. NO also helps to preserve cell viability from injury when circulation to the heart and brain is restarted after a period of cardiac arrest an no circulation. In addition, cyanide release during metabolism of SNP by the body may help protect cells by modulating the cellular metabolic rate until it too is metabolized. Cyanide metabolism is tightly regulated by the body, and enzyme processes which control cyanide metabolism could be altered as well to maximize the benefit of SNP. While SNP alone would have the negative effect of reducing the person's blood pressure, the performance of CPR serves to increase the person's blood pressure, thereby countering any negative effects induced by the administration of SNP. Another pharmacological agent, adenosine, is a potent coronary artery dilator. Administration of adenosine, or similar adenosine-like derivatives and congeners, is also effective in promoting greater perfusion to the heart, either alone or in combination with SNP or SNP-like drugs.

Performing CPR may include performing standard CPR or performing active compression decompression (ACD) CPR. The method may also include binding, manually or with an abdominal compression device, at least a portion of the person's abdomen. It may also include techniques to prevent blood flow to the legs, for example by binding the lower extremities, either continuous or in a synchronized manner with chest compressions. The method may also include at least temporarily preventing or impeding airflow to the person's lungs during at least a portion of the relaxation or decompression phase using an impedance threshold device (ITD) that is coupled with the person's airway. Such ITDs may entirely or substantially prevent or hinder respiratory gases from entering the lungs during some or all of the relaxation or decompression phase of CPR. As one specific example, an ITD may prevent respiratory gases from entering the lungs during the decompression phase until the person's negative intrathoracic pressure reaches a certain threshold, at which point a valve opens to permit respiratory gases to enter the lungs. The method may also include regulating the airflow to or from the person's lungs using an intrathoracic pressure regulator (ITPR). Such ITPRs may actively extract gases from the lungs during some or all of the relaxation or decompression phase of CPR. For example, a vacuum source may provide a continuous low-level vacuum except when a positive pressure breath is given by a ventilation source, e.g. manual or mechanical resuscitator. The applied vacuum decreases the intrathoracic pressure. Improving the artificial circulation created by the CPR may include increasing the carotid blood flow or increasing systolic and diastolic blood pressures. The method may also include stopping CPR and then restarting it multiple times, such as by, for example, in 30 second epochs for four cycles, to help preserve heart and brain function from reperfusion injury. Such a process may be referred to as stutter CPR. If stutter CPR (either ACD CPR or standard CPR) is to be performed manually, instructions and/or an aid may be provided so that the rescue personnel will have information about the sequence of delivering CPR and SNP, including in some embodiments, how to deliver the drug or drugs and perform stop/start or stutter CPR. In some cases, devices used to perform CPR may be programmed to perform stop/start or stutter CPR or have such a mode available.

Administering sodium nitroprusside may further improve a favorable characteristic of a ventricular fibrillation waveform of the person at a point in time after an onset of the cardiac arrest. Other methods can also be used to perform CPR while administering one or more vasodilator agents, with or without additional airflow controllers/manipulators/regulators, like the ITD and ITPR, to regulate the intrathoracic pressure, including those that utilize a way to compress the chest in a circumferential manner and those that provide a way to prevent reperfusion injury. These methods of CPR may further benefit from concurrent use of periodic, synchronized, or constant compression of the abdomen and/or lower extremities to help maintain most of the blood volume to the vascular spaces located anatomically above the lung diaphragms. Such methods of CPR may further enhance protection of the heart and the brain by allowing for short and periodic pauses in CPR, thereby facilitating ischemic post-conditioning, a biological process whereby the body's own defense mechanisms against injury due to poor blood flow further enhances the recovery of cell function after cardiac arrest and cardiopulmonary resuscitation. Such methods may also include the use of sodium nitroprusside and drugs like cyclosporine, which also facilitate ischemic post-conditioning.

In another embodiment, a method for increasing blood flow to vital organs during CPR using an at least partially invasive circulatory assist procedure is provided. The method may include performing CPR on a person by repetitively compressing the person's chest such that the person's chest is subject to a compression phase and a relaxation or decompression phase. The method may also include performing an at least partially invasive circulatory assist procedure on the person and administering sodium nitroprusside to the person. By administering SNP, the person's blood vessels are widened, thereby enhancing microcirculation. Further, SNP releases nitric oxide that help protect again reperfusion injury. Again, while SNP alone would have the negative effect of reducing the person's blood pressure when not in cardiac arrest, the performance of CPR serves to increase the person's blood pressure, thereby countering any negative effects induced by the administration of SNP. Methods of CPR that increase circulation more than manual CPR, such as active compression decompression CPR plus an impedance threshold device, are particularly effective with SNP. The method of CPR may also include at least a partially invasive circulatory assist procedure by inserting an intra-aortic balloon pump into the person, performing a cardiopulmonary bypass on the person or the like. Again, SNP could be used by itself or with other vasodilators such as adenosine.

In another embodiment, a method for increasing blood flow to vital organs of a person experiencing a cardiac arrest is provided. The method may include alternatively compressing and decompressing a chest of the person at a rate of about 60 to about 120 compressions/decompressions per minute to create artificial circulation. The method may also include administering sodium nitroprusside (SNP) in an amount of about 0.005 milligrams (mg) to about 5.0 mg, or in an exemplary embodiment, about 0.5 mg to about 3.0 mg, to the person to improve the artificial circulation created by the alternative compressing and lifting of the chest. SNP may be delivered as a bolus, as a continuous drip, or both. Such an approach could also include administering adenosine in a dose of about 20 mg, with a range from about 2 mg to 80 mg, either intravenously or through an intraosseal approach. The method may further include regulating inflow of respiratory gases into the person's lungs during decompressing of the chest to maintain a negative intrathoracic pressure at least below about −4.0 millimeters of Mercury (mmHg) for a time of at least about 1000 milliseconds, between positive pressure breaths. Regulating inflow of respiratory gases into the person's lungs during decompressing of the chest may include disposing a threshold valve in communication with the person's airway, wherein the threshold valve is set to open in a range from about −4.0 centimeters water (cmH20) to about −15.0 cmH20. Regulating inflow of respiratory gases into the person's lungs during decompressing of the chest may also include extracting gases from the lungs using a vacuum source. The person's lungs may experience a vacuum having a pressure of less than about −4.0 mmHg to about −12.0 mmHg. A start time of the vacuum may be substantially coincident with a start of the decompressing of the chest, and an end time of the vacuum is substantially coincident with an end of the compression of the chest. The method may moreover include binding at least a portion of the person's lower abdomen. The method may also include measuring a blood pressure of the person and altering the administration of sodium nitroprusside or a manner of chest compressions based on the blood pressure. The method may also include monitoring a physiological signal to guide the timing for defibrillation or drug administration based upon feedback from that signal or processing of the signal (for example, electrocardiography (ECG) waveform analysis).

In a further embodiment, blood flow within a patient who is in cardiac arrest is modulated or controlled to control blood flow to the heart and brain, with or without the administration of a vasodilator drug. This is done so that the vital organs receive blood in a controlled fashion. This may be particularly useful as changes in blood flow may facilitate release of endogenous vasodilators. In one aspect, blood flow is controlled or modulated so that the vital organs slowly receive additional blood over time. This may be done in a ramping fashion where the amount of blood supplied to the vital organs is slowly increased over time, or in a “stutter” fashion where blood is circulated to the vital organs for a certain time, then stopped, then again circulated. In some cases, combinations of the two methods could be used. In one specific aspect, blood circulation is controlled by stopping and starting CPR to protect against reperfusion injury by post-conditioning.

In yet other embodiments, systems and methods of the invention may include additional or other ways to compress the lower abdomen when delivering ACD CPR with or without an ITD or ITPR therapy. Such additional or other ways to compress the abdomen could be coupled with a rigid plate or board, possibly contoured, deployed under the person being treated, which could also be coupled with additional CPR devices. Merely by way of example, the rigid plate or board could include straps having a hook and loop fastener material (for example, Velcro™) or other mechanisms to provide abdominal pressure, with or without a gauge. In another example, a CPR device such as a LUCAS™ chest compression device may be coupled with the plate or board such that the plate or board provides a cradle for the for a portion of the CPR device which fits around the person. In another example a CPR device such as an AutoPulse or a load-distributing band system, could be used for chest compressions. Systems or methods of the invention could also include a defibrillator and/or a way to cool the patient, and be part of a broader “CPR workstation.” Therefore, in an exemplary embodiment, a board or plate may be inserted under the person to stabilize and support automated ACD CPR devices, as well as at least assist in maintaining abdominal compression on the person. In other embodiments, a different mechanical ACD CPR device may be used, possibly for example, the Ambu™ CardioPump™. Additionally, a defibrillator to defibrillate the person may also be coupled with the board or plate. In another embodiment, the board or plate may also be coupled with a lower extremity counter-pulsation device (discussed herein), and the lower extremity compressions could be timed with the chest compressions.

In another embodiment, a kit for increasing blood flow to vital organs during cardiopulmonary resuscitation of a person experiencing a cardiac arrest is provided. The kit may include a vasodilator drug and a mechanical device. The vasodilator drug or combination of vasodilator drugs may be provided in an amount effective to improve artificial circulation during cardiopulmonary resuscitation when administered to the person. The vasodilator drug may also be combined in the same kit with cyclosporine, a drug that facilitates post-conditioning and prevents reperfusion injury when administrated quickly after the start of CPR. The mechanical device may assist in providing cardiopulmonary resuscitation to the person. The vasodilator may, merely by way of example, be sodium nistroprusside, glycerol trinitrate, isosorbide mononitrate, isosorbide dinitrate, pentaerythritol tetranitrate, sildenafil, tadalafil, and/or vardenafil. The vasodilator may, also by way of example, be adenosine or an adenosine analog, or a methyxanthine or a methylxanthine derivative. The mechanical device may, merely by way of example, be an abdominal binding, an impedance threshold device, an intrathoracic pressure regulator, an automated chest compression device, an active compression decompression chest compression device, an electrocardiographic device, and/or a blood pressure monitor. The kit may further include a support surface. The support surface may be configured to support the person experiencing the cardiac arrest and may be coupled with the mechanical device.

In another embodiment, a method for increasing blood flow to vital organs during cardiopulmonary resuscitation of a person experiencing a cardiac arrest is provided. The method may include administering a vasodilator(s) to the person and mechanically increasing blood pressure in addition to providing cardiopulmonary resuscitation to the person.

In another embodiment, a method of increasing blood flow to the heart and brain during cardiopulmonary resuscitation of a person experiencing a cardiac arrest is provided.

The method may include using a vasodilator drug or drugs as the basis of a drug cocktail that would include one or more vasodilator drugs, and other compounds that help preserve brain cell function such as a barbiturate, cyclosporine, progesterone, other compounds that affect the neurohormonal axis, or hydrogen cyanide, in the setting of the physiological insult that results in a cardiac arrest and the marked decrease in blood flow to the brain until cardiopulmonary resuscitation is initiated.

In another embodiment, a method of inducing therapeutic hypothermia in a person is provided. The method may include administering a vasodilator to the person and lowering the temperature of the person. Administering the vasodilator to the person may include administering sodium nitroprusside to the person. Lowering the temperature of the person may include lowering the temperature of the heart or the brain of the person to between about 32° C. and about 34° C.

### BRIEF DESCRIPTION OF THE DRAWINGS

The present invention is described in conjunction with the appended figures:

FIG. 1 is a block diagram of a method of the invention for increasing blood flow to vital organs during CPR;

FIG. 2 is a block diagram of a method of the invention for increasing blood flow to vital organs during CPR using an at least partially invasive circulatory assist procedure;

FIG. 3 is a block diagram of a method of the invention for increasing blood flow to vital organs during CPR of a person experiencing a cardiac arrest;

FIG. 4 is a graph of experimental results of systolic blood pressure over time during both standard CPR and SNP enhanced ACD CPR;

FIG. 5 is a graph of experimental results of diastolic blood pressure over time during both standard CPR and SNP enhanced ACD CPR;

FIG. 6 is a graph of experimental results of carotid blood flow over time during both standard CPR and SNP enhanced ACD CPR;

FIG. 7 is a graph of experimental results of systolic blood pressure over time during both standard CPR and SNP enhanced ACD CPR;

FIG. 8 is a graph of experimental results of coronary perfusion pressure over time during both standard CPR and SNP enhanced ACD CPR;

FIG. 9 is a graph of experimental results of mean intracranial pressure over time during both standard CPR and SNP enhanced ACD CPR;

FIG. 10 is a graph of experimental results of cerebral perfusion pressure over time during both standard CPR and SNP enhanced ACD CPR;

FIG. 11 is a graph of a Fast Fourier Transformation (FFT) analysis of experimental results of ventricular fibrillation frequency and power over time during standard CPR;

FIG. 12 is a graph of a FFT analysis of experimental results of ventricular fibrillation frequency and power over time during SNP enhanced ACD CPR;

FIG. 13 is a graph of experimental results of mean ventricular fibrillation frequency and power over time during both standard CPR and SNP enhanced ACD CPR;

FIG. 14 is a graph of experimental results of carotid blood flow over time during both standard CPR and SNP enhanced ACD CPR;

FIG. 15 is a table of experimental results of hemodynamic parameters and the return of spontaneous circulation (ROSC) of both standard CPR and SNP enhanced CPR;

FIG. 16 is a table of experimental results of basic arterial blood glasses of both standard CPR and SNP enhanced CPR;

FIG. 17 is a table of experimental results of hemodynamic and respiratory parameters of both non-SNP assisted ACD CPR and SNP enhanced ACD CPR;

FIG. 18 is a table of experimental results of arterial blood gas parameters of both non-SNP assisted ACD CPR and SNP enhanced ACD CPR;

FIG. 19 is a graph of experimental results of cerebral performance category scores of both non-SNP assisted ACD CPR and SNP enhanced ACD CPR; and

FIG. 20 is an axonometric view of a person on a CPR workstation.

### DETAILED DESCRIPTION OF THE INVENTION

The ensuing description provides exemplary embodiments only, and is not intended to limit the scope, applicability or configuration of the disclosure. Rather, the ensuing description of the exemplary embodiments will provide those skilled in the art with an enabling description for implementing one or more exemplary embodiments. It being understood that various changes may be made in the function and arrangement of elements without departing from the spirit and scope of the invention as set forth in the appended claims.

Specific details are given in the following description to provide a thorough understanding of the embodiments. However, it will be understood by one of ordinary skill in the art that the embodiments may be practiced without these specific details. For example systems, processes, and other elements in the invention may be shown as components in block diagram form in order not to obscure the embodiments in unnecessary detail. In other instances, well-known processes and techniques may be discussed without unnecessary detail in order to avoid obscuring the embodiments.

Also, it is noted that individual embodiments may be described as a process which is depicted as a flowchart, a flow diagram, or a block diagram. Although a flowchart may describe the operations as a sequential process, many of the operations can be performed in parallel or concurrently. In addition, the order of the operations may be re-arranged. A process may be terminated when its operations are completed, but could have additional steps not discussed or included in a figure. Furthermore, not all operations in any particularly described process may occur in all embodiments. A process may correspond to a method, a procedure, etc. Furthermore, embodiments of the invention may be implemented, at least in part, either manually or automatically.

CPR is usually performed on a subject experiencing cardiac arrest. Cardiac arrest is the cessation of normal circulation of blood in the human body due to failure of the heart to carry out normal contractions. Essentially then, a cardiac arrest is a state of extremely low blood pressure, where because the heart is not pumping, no blood pressure is being created to push blood throughout the human body to vital organs, including, the brain and the heart itself.

The goal of CPR is to create artificial circulation, and hence blood pressure, throughout the body by rhythmic pressing on the subject's chest to manually pump blood through the heart. This artificial circulation increases oxygenated blood flow to the brain and heart, thereby increasing the chances for survival, and reducing the chances of vital organ damage, of the subject before a return of normal circulation occurs via the heart resuming normal pumping behavior, sometimes achieved via defibrillation. This resumption of normal pumping behavior is called return of spontaneous circulation (ROSC).

In the past, standard CPR techniques were very ineffective at creating meaningful blood pressure within a subject experiencing cardiac arrest. Survivors of cardiac arrest who were administered CPR often developed serious complications after the incident due to the vital organ damage experienced during the incident. Standard CPR was a simply a method to provide some, even if very little, blood pressure to the vital organs, and consequently, some small amount of oxygenated blood to those organs.

SNP is a potent vasodilator which widens blood vessels, thereby decreasing arterial and venous blood pressures and increasing microcirculation. Typically, in the past SNP has been administered to subjects experiencing severe hypertension. Subjects experiencing severe hypertension must have their blood pressure reduced immediately or risk irreversible organ damage. Because of the powerful blood pressure lowering effects of this drug, it has traditionally not been employed to assist in resuscitation efforts of a person experiencing a cardiac arrest. Since a cardiac arrest is a state of extremely low pressure in a person, and standard CPR is an emergency manual attempt that only marginally raises blood pressure, administering SNP in such cases, which has a primary effect of lowering blood pressure, has been considered counterproductive and ill-advised.

However, when combining new methods of administering both standard CPR, ACD CPR, ITD and/or ITPR technology, with delivery of SNP, such methods act synergistically to increase blood flow and pressure created by the CPR methods. Mechanical (via CPR) and pharmacological (via SNP) modulation of regional vascular resistances combine in a unique way to improve, in a manner unknown until now, resuscitation outcomes of CPR. Specifically, the methods described herein create an improved chance of return of spontaneous circulation (ROSC), as well as 24-hour neurological intact survival when compared to prior methods known in the art for performing CPR. Thus the systems and the methods of the invention counter-intuitively provide for administration of SNP, a drug which normally decreases blood pressure, during cardiac arrests and other heart failures characterized by critically low blood pressure, to achieve beneficial results.

In one embodiment of the invention, a method for increasing blood flow to vital organs during CPR of a person experiencing cardiac arrest is provided. The method proceeds by performing CPR on a person to create artificial circulation by repetitively compressing the person's chest such that the person's chest is subject to a compression phase and a relaxation or decompression phase. The method may also include administering a vasodilator, such as sodium nitroprusside, to the person (SNP enhanced CPR) to improve the artificial circulation created by the CPR. The method may also include administering SNP together with another vasodilator such as adenosine. These two agents may act synergistically to dilate blood vessels to and in the heart and brain, thereby increasing blood perfusion of these vital organs during cardiopulmonary resuscitation.

By administering SNP and/or adenosine (or other vasodilator), the person's blood vessels are dilated, thereby enhancing microcirculation. While SNP alone would have the negative effect of reducing the person's blood pressure, the performance of CPR serves to increase the person's blood pressure, thereby countering any negative effects induced by the administration of SNP. Possible specific benefits of SNP administration during CPR may also include increasing the carotid blood flow or increasing systolic and diastolic blood pressures. Another possible benefit may be improving favorable characteristics of a ventricular fibrillation waveform of the person at a point in time after an onset of the cardiac arrest.

In some embodiments, SNP may be administered to a person during the post-resuscitation phase to assist in stabilizing circulation. Dosage may be about 0.01 mg of SNP bolus, followed by an intravenous drip. This may decrease the development of cardiac dysfunction, pulmonary edema, and poor organ perfusion. Likewise, use of SNP during CPR may be used to reverse pulmonary edema and fluid build up in the lungs, and promote forward blood flow by lowering resistance within or dilating the arterial vascular beds.

Performing CPR may include performing standard CPR or performing ACD CPR. Performing standard CPR may include traditional methods of manual chest compression and mouth-to-mouth respiration, or methods which employ automatic devices such as automated pistons, inflatable vests, or the like, for chest compression and ventilator bags for respiration.

Systems and methods for performing ACD CPR are discussed in U.S. Provisional Patent Application Ser. No. 61/304,148 by Greg Voss et al. entitled “GUIDED ACTIVE COMPRESSION DECOMPRESSION CARDIOPULMONARY RESUSCITATION SYSTEMS AND METHODS” filed on Feb. 12, 2010; U.S. Pat. No. 5,454,779 to Keith G. Lurie et al. entitled, “DEVICES AND METHODS FOR EXTERNAL CHEST COMPRESSION” issued on Oct. 3, 1995; and U.S. Pat. No. 5,645,522 to Keith Lurie et al. entitled “DEVICES AND METHODS FOR CONTROLLED EXTERNAL CHEST COMPRESSION” issued on Jul. 8, 1997, the entire contents of which are all hereby incorporated by reference, for all purposes, as if fully set forth herein.

In some embodiments, the method may also include stimulating the phrenic nerve, either manually or automatically, in order to reduce intrathoracic pressures during the decompression phase of CPR. Systems and methods for stimulating the phrenic nerve are discussed in U.S. Pat. No. 6,463,327 to Keith G. Lurie et al. entitled “STIMULATORY DEVICE AND METHODS TO ELECTRICALLY STIMULATE THE PHRENIC NERVE” issued on Oct. 8, 2002, the entire contents of which is hereby incorporated by reference, for all purposes, as if fully set forth herein.

The method may also include binding, manually or with a compression device, at least a portion of the person's abdomen or extremities. Systems and methods for binding a person's extremities are discussed in U.S. Patent Application Publication No. 2009/0062701 by Demetris Yannopoulos et al. entitled “LOWER EXTREMITY COMPRESSION DEVICES, SYSTEMS AND METHODS TO ENHANCE CIRCULATION” published on Mar. 5, 2009, the entire contents of which is hereby incorporated by reference, for all purposes, as if fully set forth herein. Such binding may be particularly useful in increasing the person's blood pressure in the thorax and/or upper body to counter any negative effects produced by administering SNP.

The effectiveness of CPR in increasing blood flows may be enhanced by using an intrathoracic pressure device (ITD) or an intrathoracic pressure regulator (ITPR) device, which is typically connected with a ventilator. Systems and methods regarding ITD's and ITPR's are discussed in U.S. Provisional Patent Application Ser. No. 61/218,763 by Keith G. Lurie et al. entitled “VACUUM AND POSITIVE PRESSURE VENTILATION SYSTEMS AND METHODS FOR INTRATHORACIC PRESSURE REGULATION” filed on Jun. 19, 2009; U.S. Patent Application Publication No. 2003/0062040 by Keith G. Lurie et al. entitled ‘FACE MASK VENTILATION/PERFUSION SYSTEMS AND METHOD” published on Apr. 3, 3003; U.S. Patent Application Publication No. 2007/0221222 by Keith Lurie entitled “CPR DEVICES AND METHODS OF UTILIZING A CONTINUOUS SUPPLY OF RESPIRATORY GASES” published on Sep. 27, 2007; U.S. Patent Application Publication No. 2007/0277826 by Keith G. Lurie entitled “SYSTEMS AND METHODS FOR MODULATING AUTONOMIC FUNCTION” published on Dec. 6, 2007; U.S. Patent Application Publication No. 2008/0255482 by Keith G. Lurie entitled “INTRATHORACIC PRESSURE LIMITER AND CPR DEVICE FOR REDUCING INTRACRANIAL PRESSURE AND METHODS OF USE” published on Oct. 16, 2008; U.S. Patent Application Publication No. 2009/0020128 by Anja Metzger et al. entitled “METHOD AND SYSTEM TO DECREASE INTRACRANIAL PRESSURE, ENHANCE CIRCULATION, AND ENCOURAGE SPONTANEOUS RESPIRATION” published Jan. 22, 2009; U.S. Patent Application Publication No. 2009/0277447 by Greg Voss et al. entitled “SYSTEM, METHOD, AND DEVICE TO INCREASE CIRCULATION DURING CPR WITHOUT REQUIRING POSITIVE PRESSURE VENTILATION” published Nov. 12, 2009; U.S. Pat. No. 7,204,251 to Keith G. Lurie entitled “DIABETES TREATMENT SYSTEMS AND METHODS” issued on Apr. 17, 2007; U.S. Pat. No. 6,425,393 to Keith G. Lurie et al. entitled “AUTOMATIC VARIABLE POSITIVE EXPIRATORY PRESSURE VALVE AND METHODS” issued on Jul. 30, 2002; U.S. Pat. No. 6,155,257 to Keith G. Lurie et al. entitled “CARDIOPULMONARY RESUSCITATION VENTILATOR AND METHODS” issued on Dec. 5, 2000; U.S. Pat. No. 5,692,498 to Keith G. Lurie et al. entitled, “CPR DEVICE HAVING VALVE FOR INCREASING THE DURATION AND MAGNITUDE OF NEGATIVE INTRATHORACIC PRESSURES” issued on Dec. 2, 1997; and U.S. Pat. No. 5,551,420 to Keith G. Lurie et al. entitled, “CPR DEVICE AND METHOD WITH STRUCTURE FOR INCREASING THE DURATION AND MAGNITUDE OF NEGATIVE INTRATHORACIC PRESSURES” issued on Sep. 3, 1996, the entire contents of which are all hereby incorporated by reference, for all purposes, as if fully set forth herein.

ITD's and ITPR's operate to increase or regulate the magnitude of negative intrathoracic pressure during the decompression or relaxation phase of CPR. For example, ITDs may entirely or substantially prevent or hinder respiratory gases from entering the lungs during some or all of the relaxation or decompression phase of CPR to increase the amount and/or duration of the person's negative intrathoracic pressure. As one specific example, an ITD may prevent respiratory gases from entering the lungs during the decompression phase until the person's negative intrathoracic pressure reaches a certain threshold, at which point a valve opens to permit respiratory gases to enter the lungs. ITPRs may be used to regulator respiratory or other gas flow into and out of the patient's lungs. For instance, an ITPR may actively extract gases from the lungs during some or all of the relaxation or decompression phase of CPR. For example, a vacuum source may provide a continuous low-level vacuum. In some cases, the low-level vacuum may be interrupted when a positive pressure breath is given by a ventilation source, e.g., manual or mechanical resuscitator. The applied vacuum decreases the intrathoracic pressure, particularly during the decompression or relaxation phase of CPR. Hence, by using an ITD and/or ITPR, blood flow back to the thorax is increased so that during the next chest compression, more blood is available to be circulated by throughout the body from the thorax. In addition, ITD's and ITPR's at least partially assist in lowering intracranial pressure. Hence, even if the administration of SNP serves to reduce a person's blood pressure, the use of an ITD and/or ITPR serves to increase blood flow so that advantage can be taken of SNP's potent vasodilatory effects.

In some embodiments, the method may further include measuring a blood pressure of the person. The administration of sodium nitroprusside, or a manner of the delivered chest compressions, may be altered based on the measured blood pressure. In these or other embodiments, the method may also include ventilating the person, at least periodically. In some of these embodiments, the ventilation of the person may be altered based on the measured blood pressure. In addition, circulatory assist devices in the form of extracorporeal membrane oxygenators can be used with SNP in the treatment of patients experiencing cardiac arrest, as can invasive circulatory assist devices without membrane oxygenators.

In another embodiment of the invention, a method for increasing blood flow to vital organs during CPR using an at least partially invasive circulatory assist procedure is provided. The method may include performing CPR on a person by repetitively compressing the person's chest such that the person's chest is subject to a compression phase and a relaxation or decompression phase. The method may also include performing an at least partially invasive circulatory assist procedure on the person and administering sodium nitroprusside to the person. The at least partially invasive circulatory assist procedure may include inserting an intra-aortic balloon pump into the person or performing a cardiopulmonary bypass on the person.

Invasive circulatory assist procedures may increase blood flow and/or blood pressure via different means. Merely by way of example, intra-aortic balloon pumps may be surgically inserted into the aorta and actively deflate in systole, thereby decreasing the amount of pressure the muscles of the heart must exert to deliver the same amount of blood flow. Additionally, the intra-aortic balloon pump may actively inflate in diastole, thereby increasing the amount of oxygenated blood flow to the coronary arteries which power the heart. As another example, a cardiopulmonary bypass is a technique whereby a machine is used to take over the functions of a person's heart and lungs to deliver pressurized flow of oxygenated blood to the body during surgery.

In another embodiment, a method for increasing blood flow to vital organs of a person experiencing a cardiac arrest is provided. The method may include alternatively compressing and decompressing a chest of the person at a rate of about 60 to about 120 compressions/decompressions per minute to create artificial circulation. The method may also include administering sodium nitroprusside (SNP) in an amount of about 0.005 mg to about 5.0 mg, or in an exemplary embodiment, about 0.5 mg to about 3.0 mg, to the person to improve the artificial circulation created by the alternative compressing and lifting of the chest. SNP may be delivered as a bolus, as a continuous drop, or both. The method may further include regulating inflow of respiratory gases into the person's lungs during decompressing of the chest to maintain a negative intrathoracic pressure at least below about −4.0 mmHg for a time of at least about 1000 milliseconds between positive pressure breaths.

Regulating inflow of respiratory gases into the person's lungs during decompressing of the chest may include disposing a threshold valve in communication with the person's airway, wherein the threshold valve is set to open in a range from about −4.0 cmH20 to about −15.0 cmH20. Regulating inflow of respiratory gases into the person's lungs during decompressing of the chest may also include extracting gases from the lungs using a vacuum source. The person's lungs may experience a vacuum having a pressure of less than about −4.0 mmHG to about −12.0 mmHG. A start time of the vacuum may be substantially coincident with a start of the decompressing of the chest, and an end time of the vacuum is substantially coincident with an end of the compression of the chest.

The method may moreover include binding at least a portion of the person's lower abdomen. The method may also include measuring a blood pressure of the person and altering the administration of sodium nitroprusside or a manner of chest compressions based on the blood pressure. The method may also include monitoring a physiological signal to guide the timing for defibrillation or drug administration based upon feedback from that signal or processing of the signal (for example, electrocardiography (ECG) waveform analysis).

In each of these embodiments, SNP or a SNP-like drug could be used by itself, or in combination with another vasodilator, for example adenosine or an adenosine analog. A combination of vasodilator agents has the potential dilate the cerebral and coronary artery vasculature to a greater degree at an overall lower dose than a single compound by itself.

Another feature of the invention is the ability to control or modulate blood flow within a patient who is in cardiac arrest, and in particular, to control blood flow to the heart and brain, with or without the administration of a vasodilator drug, such that the vital organs receive blood in a controlled fashion. This may be particularly useful as changes in blood flow may facilitate release of endogenous vasodilators. More specifically, blood flow is controlled or modulated so that the vital organs slowly receive additional blood over time. This may be done in a ramping fashion where the amount of blood supplied to the vital organs is slowly increased over time, or in a “stutter” fashion where blood is circulated to the vital organs for a certain time, then stopped, then again circulated. In some cases, combinations of the two methods could be used.

Merely by way of example, blood circulation may be ramped up over time so that initially the blood flow to the vital organs may be about 5% to 100% of what a healthy person may expect to receive with normal heart function. Over a time period of about <1 minute to over an hour, the circulation may be increased so that the blood flow to the vital organs is about 5% to about 100% and even higher of what a healthy person may expect to receive. The ramping function could be linear, non-linear, or may jump in discrete steps.

For the “stutter” process, blood may be circulated to the vital organs for set start and stop times, such as by causing circulation for about 40 seconds, and then stopping circulation for about 20 seconds, and then resuming circulation for 40 about seconds, and then stopping circulation for about 20 seconds, etc. The time intervals where circulation occurs and is stopped could remain the same, or could vary over time. For example, the time during which circulation occurs could increase over time. The time during which circulation is stopped could also vary over time, such as by decreasing the length of the stopping periods over time.

Blood circulation may be facilitated in a variety of ways, including manual or automated CPR or ACD CPR, external or internal blood pumps, pressure cuffs, lateral gravity (g) acceleration, and the like. Software programs may be employed to control circulation devices so that blood circulation to the vital organs may be controlled as just described. Similarly, manual instructions may be provided to rescuers performing manual blood circulation techniques, such as standard CPR. Further, instructions may be provided for when to administer a vasodilator drug during the process, if appropriate.

As one specific example, the effectiveness of CPR with SNP or a SNP-like drug can be further enhanced by providing CPR for a period of time, for example 40 seconds, and then stopping for 20 seconds, and then resuming CPR for 40 seconds, and then stopping CPR for 20 seconds, and then resuming CPR. Use of between 0.05 and 1 mg of epinephrine during this post-conditioning process can be used to further improve circulation of blood flow to the heart and brain and long-term neurologically-intact survival rates. If such a “stutter” CPR process (either ACD CPR or standard CPR) is to be performed manually, a kit may be provided with instructions and/or a mechanical aid so that the rescue personnel will have information about the sequence of delivering CPR and SNP, including in some embodiments, how to deliver the drug or drugs and perform stop/start or stutter CPR. In some cases, CPR will be performed using a mechanized device, and such devices used to perform CPR may be programmed to perform stop/start or stutter CPR or have such a mode available.

One specific example of such a method is based on the observation that mechanical post conditioning (PC) with intermittent initiation of flow (“stutter” reperfusion) has been shown to decrease infarction size in ST elevation infarction and decrease ischemic stroke size in animals. An experiment was performed to determine whether when using sodium nitroprusside-enhanced (SNPeCPR) cardiopulmonary resuscitation (CPR), mechanical post conditioning with stutter CPR (20-second CPR pauses), begun immediately on SNPeCPR initiation, improves 24-hour cerebral function compared to 12 hours of therapeutic hypothermia (TH) post resuscitation.

This was shown in an experiment using 14 anesthetized and intubated pigs that underwent 15 minutes of untreated VF followed by 5 minutes of SNPeCPR comprised of active compression decompression CPR plus an inspiratory impedance threshold device combined with abdominal binding. The ITD prevented respiratory gases from entering the lungs during the decompression phase of CPR. In this example, the ITD had a safety check valve that would allow for inspiration when the intrathoracic pressure was less than minus 16 cm H2O, which does not occur during CPR. The abdomen in this example was compressed with 40-50 lbs of pressure, applied continuously with a bend human arm, using the forearm and a force gauge. Further, 2 mg of sodium nitroprusside (SNP) were given IV at minute 1 and 1 mg at minute 3 of CPR. All animals received in addition 0.5 mg of epinephrine at minute 5, 30 seconds before the first defibrillation attempt. Six animals (PC group), were treated with 40 seconds of SNPeCPR and the first dose of SNP were followed by 20-second pauses (cessation of perfusion) and 20 second of SNPeCPR for at total of 4 cycles for up to 3 minutes. After that animals had uninterrupted SNPeCPR until defibrillation at minute 5. The other 8 animals (TH group) had SNPeCPR for a total of 5 minutes without interruptions. The TH group received 12 hours of TH (core temp=33° C.). The PC group received no TH. Cerebral performance was scored at 24 hours by a veterinarian blinded to the treatment group.

During SNPeCPR, there were no hemodynamic differences except for a significantly higher aortic pressure response to epinephrine at min 5 (SBP/DBP; 148±12/78±7 in the PC group versus 110±9/62±5 mmHg in the TH group, p<0.05). Return of spontaneous circulation rates and 24-hour survival was 100% for both groups. CPC was significantly lower in the PC group (1±0) versus the TH group (2.4±0.8), p<0.01. Hence, in this porcine model of cardiac arrest and SNPeCPR, mechanical PC with pauses in compressions at the initiation of the resuscitation efforts prevented 24-hour neurological dysfunction after 15 minutes of untreated VF and was superior to TH.

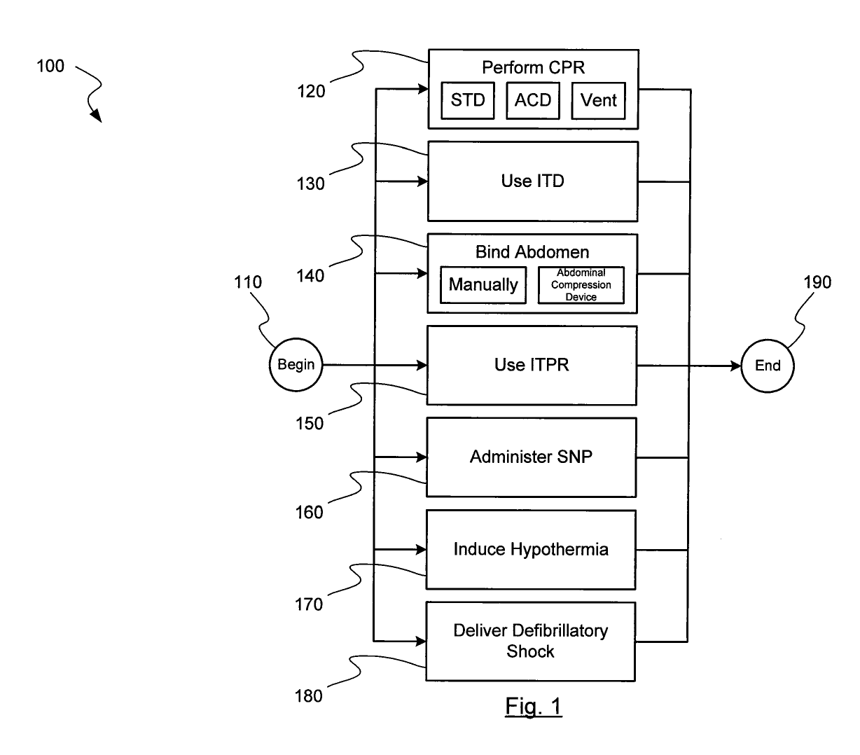

Turning now to FIG. 1, a block diagram of a method 100 of the invention for increasing blood flow to vital organs during CPR is shown. At block 110, method 100 may begin, and at block 120 CPR may be performed. Either standard CPR or ACD CPR may be performed, possibly using any of the techniques described herein. However, ACD CPR may be more effective. This serves to circulate blood throughout the person by compressing the chest and/or creating a negative intrathoracic pressure to draw blood back into the chest cavity.

At block 130, possibly concurrent with other steps of method 100, airflow to the subject's lungs may at least be temporarily prevented or impeded during at least a portion of the relaxation or decompression phase of the CPR using an impedance threshold device (ITD) that is coupled with the person's airway. As discussed above, an ITD operates to increase the magnitude of negative intrathoracic pressure and assist in flowing blood back to the thorax during a CPR decompression or relaxation, so that the next compression produces greater blood flow out of the heart to the vital organs. One example of an ITD is the ResQPOD™ ITD available from Advanced Circulatory Systems, Inc. of Roseville, Minn. Other ITD's described herein may also be used.

At block 140, possibly concurrent with other steps of method 100, the abdomen, or other extremities, of the subject may be bound. Either manual binding of the abdomen or extremities, or an abdominal or extremity compression device, may be employed. This helps to increase the person's blood pressure to vital organs by limiting blood flow to the extremities, thereby increasing the blood flow to the thorax. Prior to the instant invention, binding a persons abdomen during CPR may have been counterproductive because of the high blood pressures it created in various portions of the upper body. However, through the use of SNP as described herein, use of an abdominal binding may improve CPR outcomes.

At block 150, possibly concurrent with other steps of method 100, airflow to or from the person's lungs may be regulated using an ITPR device. Examples of ITPR devices include ResQVent™ and CirQLato™ available from Advanced Circulatory Systems, Inc., as well as other systems discussed supra.

At block 160, possibly concurrent with other steps of method 100, SNP may be administered to the subject. SNP may be dosed at different levels and at different intervals or rates, possibly dependent on characteristics of the subject or their current medical condition.

Merely by way of example, SNP may be administered continuously via intravenous delivery, possibly with other medicines. In other embodiments, SNP may be administered in doses, possibly via hypodermic injection. Merely by way of example, in some embodiments, method 100 may include administering two or more doses of SNP with at least a substantially 5 minute interval between the doses. In many embodiments, the first dose of SNP may occur during the very early stages of method 100, possibly within one or two of the initial minutes of beginning CPR chest compressions, which may possibly commence with the subject experiencing cardiac arrest.

Dosage of SNP may be based on various characteristics of the subject, including possibly weight or mass of the subject. Merely by way of example, 1 milligram (mg) of SNP may be administered to the subject per 30 kilograms of mass of the person (1 mg per 66 pounds of weight of the person). In other embodiments, amounts greater than 1 milligram per similar mass or weight of subject may be administered. In some embodiments, intracoronary administration of SNP may also be employed. In addition, other nitric oxide donor drugs (for example, nitroglycerin) may be used, as well as other vasodilator drugs such a hydralzaine or Viagra™, adenosine antagonists, calcium channel antagonists, and beta-adrenergic blockers, as well as inhaled nitric oxide.

At block 170, possibly concurrent with other steps of method 100, therapeutic hypothermia (TH) may be induced in the subject. For example, the person's body temperature may be reduced to between about 32 and about 34 degrees Celsius (about 90 and about 93 degree Fahrenheit).

TH improves mortality and neurological outcomes when applied after resuscitation. Additionally, the neurological and cardiac benefits are magnified when it is applied during CPR and the target temperature is achieved as soon as possible. While it is most ideal to cool the patient before CPR is started, that is usually not feasible.

Unfortunately, most intra CPR cooling methods are either invasive or non effective for the large thermal mass of the human body. Intravenous cold saline when given with standard CPR causes severe reduction of the coronary perfusion pressure by increasing the right atrial pressure and therefore has negative effect to resuscitation rates. External cooling methods (for example, cooling blanket, etc.) have limited efficacy during CPR because of the intense cutaneous (skin) vasoconstriction due to high catecholamine levels. Hence it may take many hours (up to 6-8 hours) to achieve the target temperature (32-34° C.).

Heat exchange can be maximized by increasing the contact surface with the coolant, by increasing blood flow for a given cold saline volume, and by divesting cold blood to the tissues needed. The primary target of such cooling is the brain. Embodiments of the invention which employ SNP during various forms of CPR optimize blood flow levels to at least near normal levels for vital organs, and therefore can improve coronary perfusion to such a degree that addition of intravenous (or otherwise delivered) cold saline does not cause significant reduction of coronary perfusion pressure. Additionally, because an abdominal binding may be employed, colder blood flow via cold saline will be more likely diverted to vital organs such as the heart and brain, rather than to extremities where a lower temperature is not required.

Finally, SNP causes skin vasodilatation, increasing upper body skin perfusion and allowing conductive and convective heat transfer means applied external to the body to be more effective (for example, cooling liquids, ice, cooling gels, cooling blankets, liquid misting, evaporative cooling via alcohol or other fast evaporating liquid, etc.). The above methods may allow a target TH temperature to be reaches at the heart and brain within 5 to 10 minutes after TH administration. Additionally, because of increased skin perfusion, faster drug administration may occur via intravenous, subcutaneous, and/or other delivery methods. In some embodiments, SNP may be used post resuscitation to increase the effectiveness of cooling during post resuscitation TH.

At block 180, possibly concurrent with other steps of method 100, a defibrillatory shock may be delivered to the subject. Systems and methods for delivering defibrillatory shocks are discussed in U.S. Provisional Patent Application No. 60/917,602 by Keith G. Lurie et al. entitled “METHOD AND SYSTEM TO TREAT PATIENTS IN CARDIAC ARREST USING VENTRICULAR DEFIBRILLATION” filed on May 11, 2007, the entire contents of which is hereby incorporated by reference, for all purposes, as if fully set forth herein. At block 190, method 100 may end.

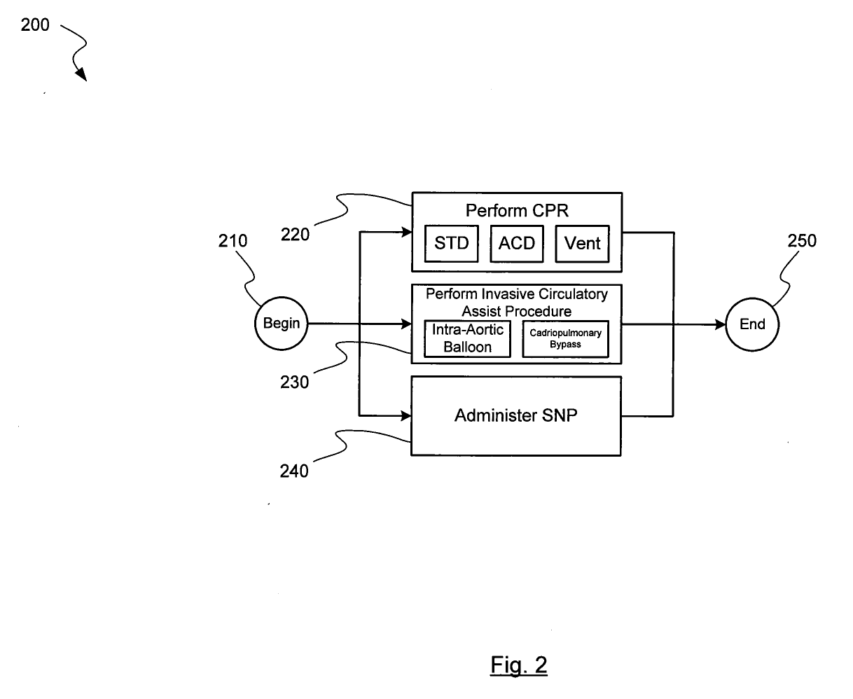

FIG. 2 shows a block diagram of a method 200 of the invention for increasing blood flow to vital organs during CPR using an at least partially invasive circulatory assist procedure. At block 210, method 200 may begin, and at block 220 CPR may be performed. Either standard CPR or ACD CPR may be performed.

At block 230, possibly concurrent with other steps of method 200, an invasive circulatory assist procedure may be performed on the subject. Two examples of invasive circulatory assist procedures are intra-aortic balloon insertion and cardiopulmonary bypass.

At block 240, possibly concurrent with other steps of method 200, SNP may be administered to the subject. SNP may be dosed at different levels and at different intervals or rates, possibly dependent on characteristics of the subject or their current medical condition, as discussed elsewhere herein.

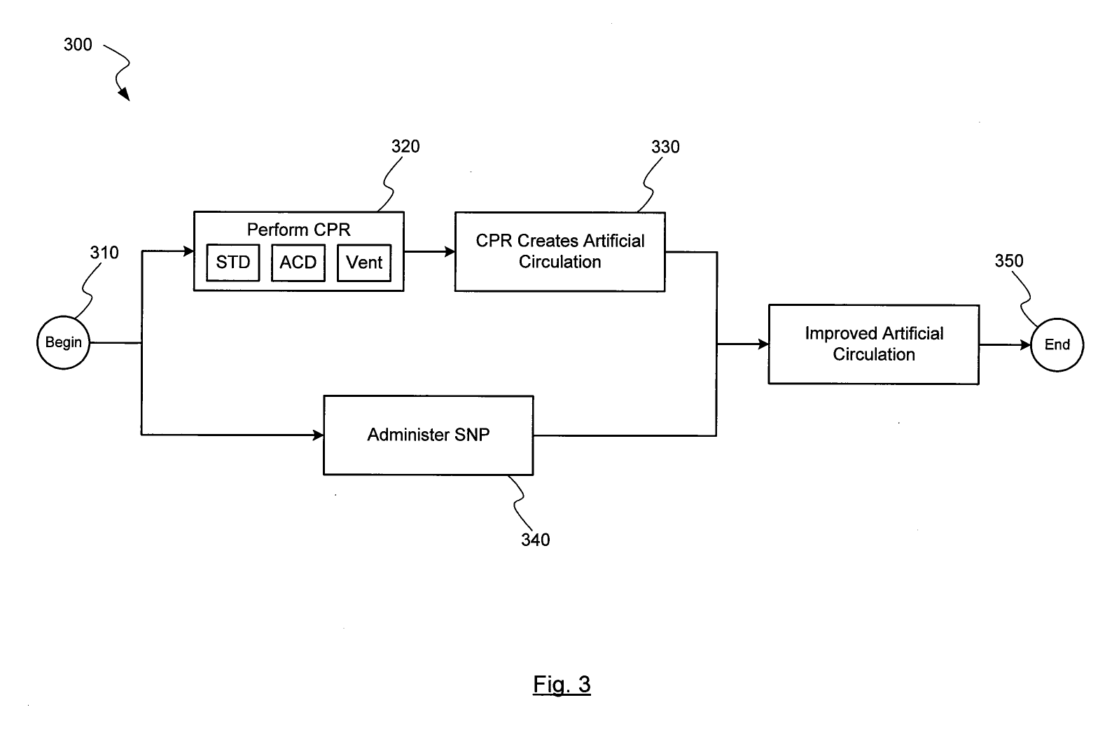

FIG. 3 shows a block diagram of a method 300 of the invention for increasing blood flow to vital organs during CPR of a person experiencing a cardiac arrest. At block 310, method 300 may begin, and at block 320 CPR may be performed. Either standard CPR or ACD CPR may be performed. Standard CPR or ACD CPR may employ chest compressions at rates between about 60 and about 120 compressions per minute. Additionally, standard CPR or ACD CPR may include periodic ventilating at rates between about 6 and about 20 breaths per minute, and in an exemplary embodiment, at about 10 breaths per minute, and at a 10 milliliter per kilogram (mL/kg) tidal volume (or a range of about 4 mL/kg to about 15 mL/kg).

At block 330, the CPR may create artificial circulation in the subject. At block 340, possibly concurrent with other steps of method 300, SNP may be administered to the subject. SNP may be dosed at different levels and at different intervals or rates, possibly dependent on characteristics of the subject or their current medical condition, as discussed elsewhere herein.

The graphs and tables discussed below show experimental results of various CPR methods, with and without SNP enhancement, as applied to 30 kilogram (plus or minus 1 kilogram) Yorkshire female farm bread pigs after 6 minutes of untreated induced ventricular fibrillation (VF). Chest compressions were administered via a pneumatically driven automatic piston device (Pneumatic Compression Controller, Ambu International, Glostrup, Denmark). The compression rate was 100 compressions/min uninterrupted, with a 50% duty cycle and a compression depth of 25% of the anterior-posterior chest diameter. An ITD was used during ACD CPR processes. The ITD used in these studies prevented respiratory gases from entering the lungs during the decompression phase of CPR. In these studies, the ITD had a safety check valve that would allow for inspiration when the intrathoracic pressure was less than minus 16 cm H2O, which does not occur during CPR. During CPR, positive-pressure ventilations were delivered asynchronously, to simulate advanced life support with a manual resuscitator bad (Smart Bag, O2 Systems, Toronto, Ontario Canada). The fraction of inspired oxygen was 1.0, the tidal volume was 320 cc and the respiratory rate was 8-10 breaths/min.

In a first portion of the experiments, eight pigs were subject to standard CPR and defibrillation versus eight pigs which received progressive treatment involving (1) standard CPR, (2) the addition of SNP, (3) ACD CPR, (4) the addition of ITD use, (5) the addition of an abdominal binding (AB), and (6) debilitation. In a second portion of the experiments, five pigs received standard CPR and defibrillation versus seven pigs which received ACD CPR, ITD, AB, SNP, and defibrillation. In a third portion of the experiments, eight pigs received ACD CPR, ITD, AB and defibrillation versus eight pigs which received ACD CPR, ITD, AB, SNP, and defibrillation.

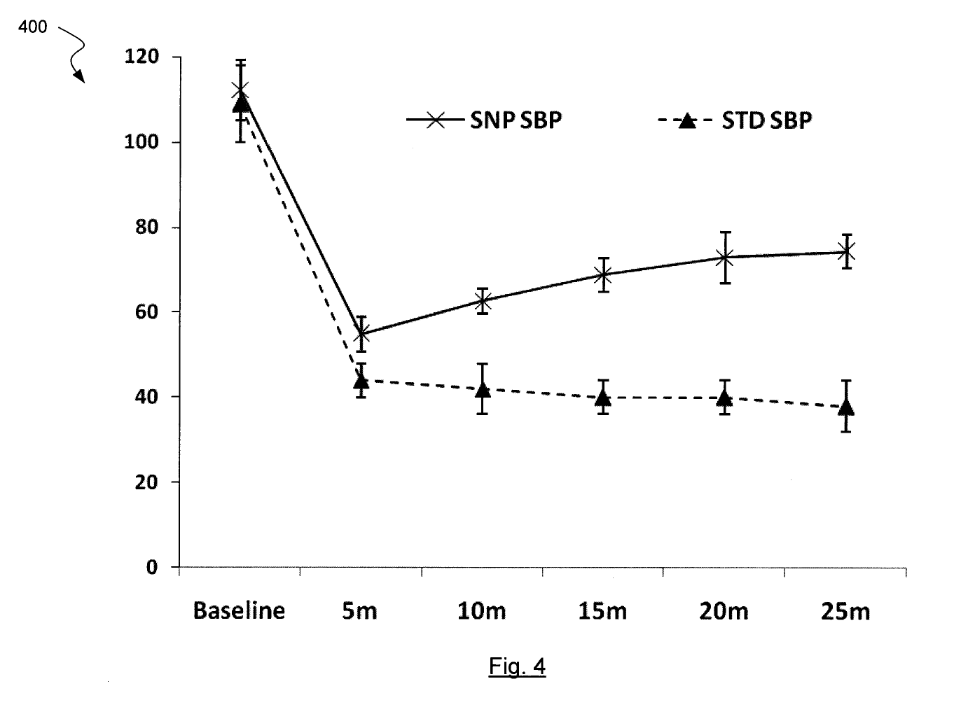

FIG. 4 shows a graph 400 of experimental results of systolic blood pressure (SBP) over time during both standard CPR and SNP enhanced ACD CPR (including ITD and abdominal binding) The abdomen was compressed with 40-50 lbs of pressure, applied continuously with a bend human arm, using the forearm and a force gauge. The SBP produced by the SNP enhanced CPR (SNP SBP) is statistically significantly increased over the SBP produced by standard CPR (STD SBP) with a p-value of less than 0.05. Improved SBP using SNP enhanced ACD CPR over standard CPR results in greater blood flow to vital organs, improving the probability of ROSC and decreasing the likelihood and amount of damage to vital organs during CPR.

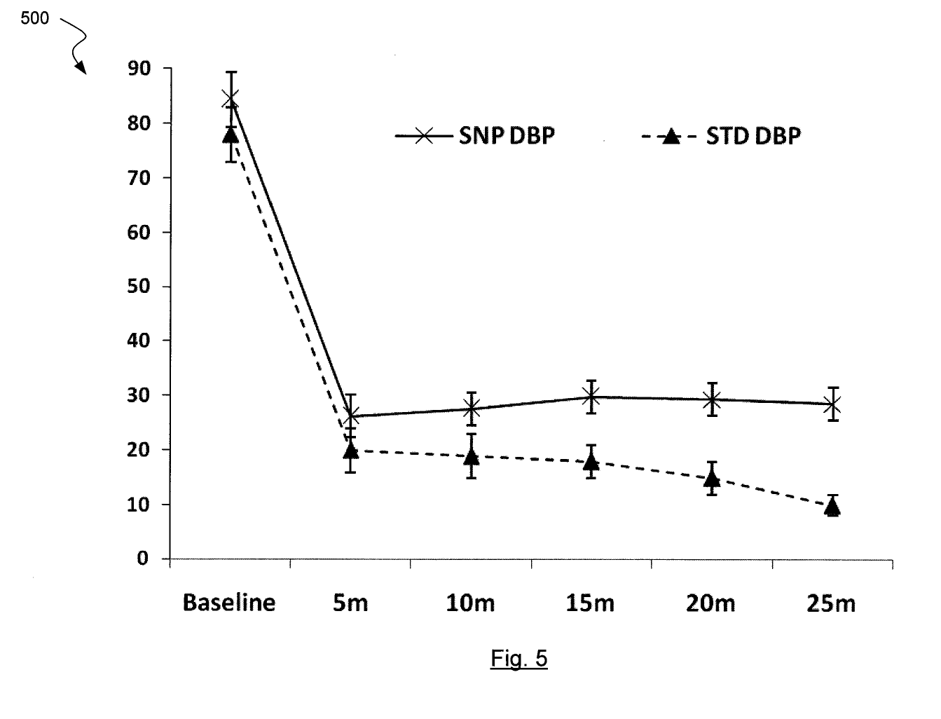

FIG. 5 shows a graph 500 of experimental results of diastolic blood pressure (DBP) over time during both standard CPR and SNP enhanced ACD CPR (including ITD and abdominal binding). The DBP produced by the SNP enhanced CPR (SNP DBP) is statistically significantly increased over the DBP produced by standard CPR (STD DBP) with a p-value of less than 0.05. Improved DBP using SNP enhanced ACD CPR over standard CPR results in greater blood flow to vital organs, improving the probability of ROSC and decreasing the likelihood and amount of damage to vital organs during CPR.

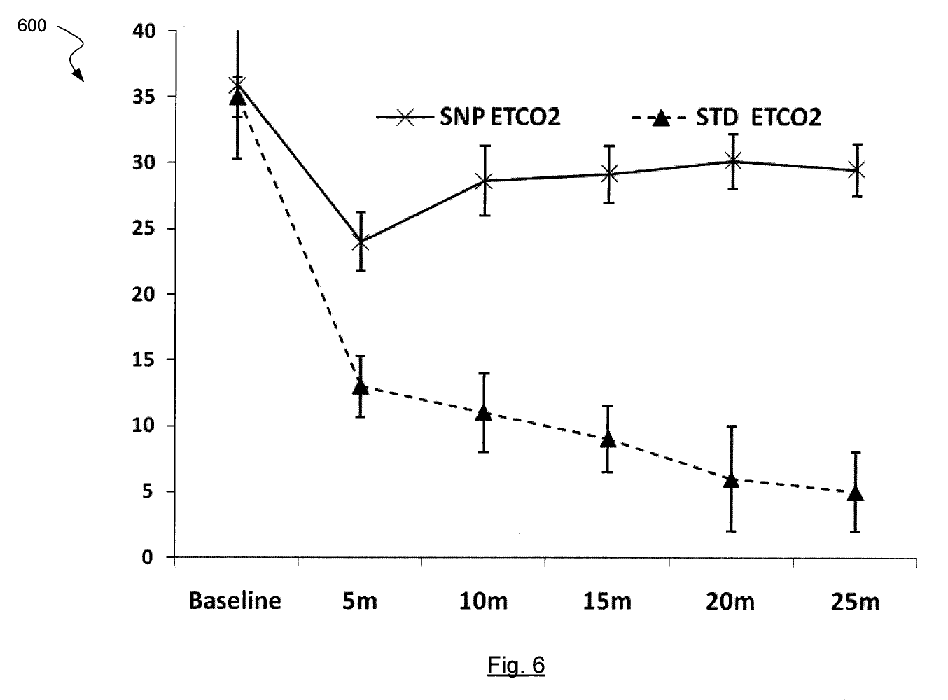

FIG. 6 shows a graph 600 of experimental results of end tidal carbon dioxide (EtCO₂) over time during both standard CPR and SNP enhanced ACD CPR (including ITD and abdominal binding). The EtCO₂ produced by the SNP enhanced CPR (SNP EtCO₂) is statistically significantly increased over the EtCO₂ produced by standard CPR (STD EtCO₂) with a p-value of less than 0.05. EtCO₂, a surrogate marker for circulation, was improved using SNP enhanced ACD CPR over standard CPR, and is reflective of greater cardiac output generally and more specifically increased pulmonary blood flow, which improves the probability of ROSC and decreases the likelihood and amount of damage to vital organs during CPR.

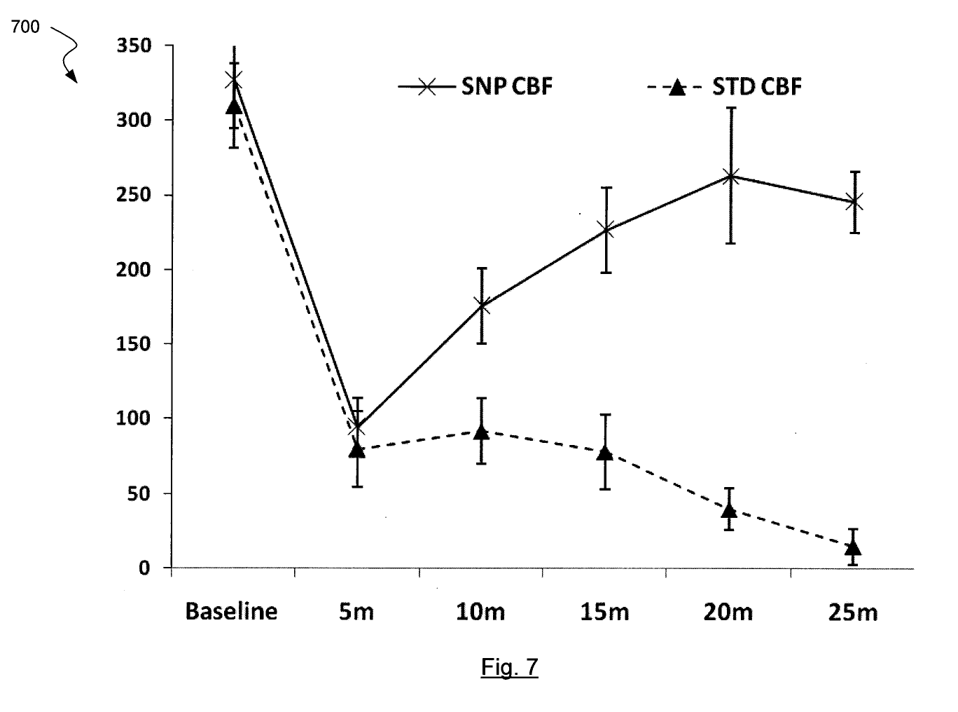

FIG. 7 shows a graph 700 of experimental results of carotid blood flow (CBF) over time during both standard CPR and SNP enhanced ACD CPR (including ITD and abdominal binding). Beginning at ten minutes, the CBF produced by the SNP enhanced CPR (SNP CBF) is statistically significantly increased over the CBF produced by standard CPR (STD CBF) with a p-value of less than 0.05. Improved CBF using SNP enhanced ACD CPR over standard CPR results in greater blood flow to the head and brain, decreasing the likelihood and amount of neurological damage during CPR.

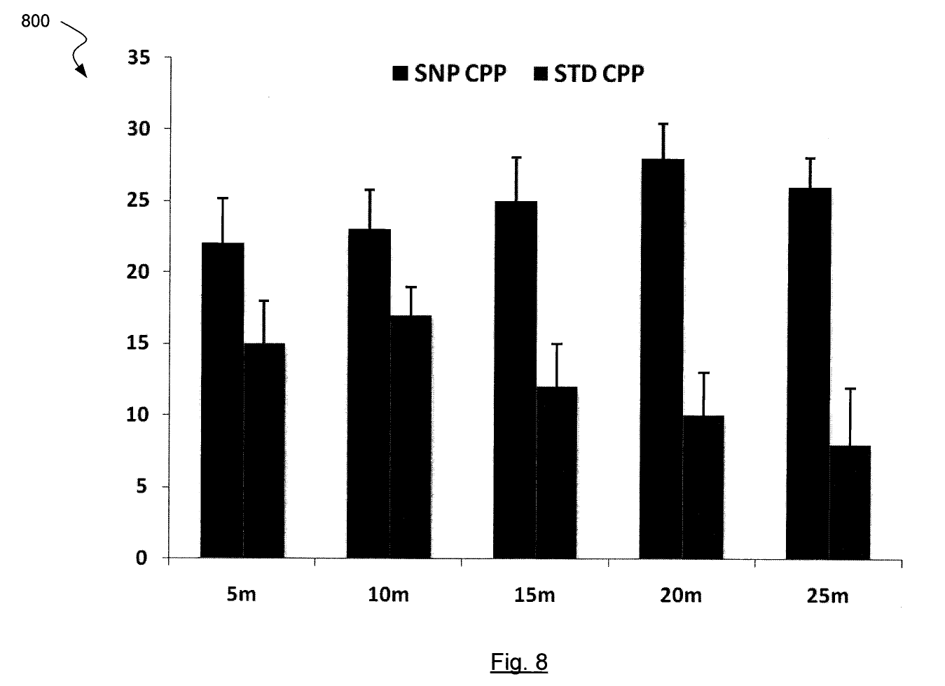

FIG. 8 shows a graph 800 of experimental results of coronary perfusion pressure (CPP) over time during both standard CPR and SNP enhanced ACD CPR (including ITD and abdominal binding). The CPP produced by the SNP enhanced CPR (SNP CPP) is statistically significantly increased over the CPP produced by standard CPR (STD CPP) with a p-value of less than 0.05. Improved CPP using SNP enhanced ACD CPR over standard CPR results in greater blood flow to the myocardium, improving the probability of ROSC and decreasing the likelihood and amount of damage to vital organs during CPR.

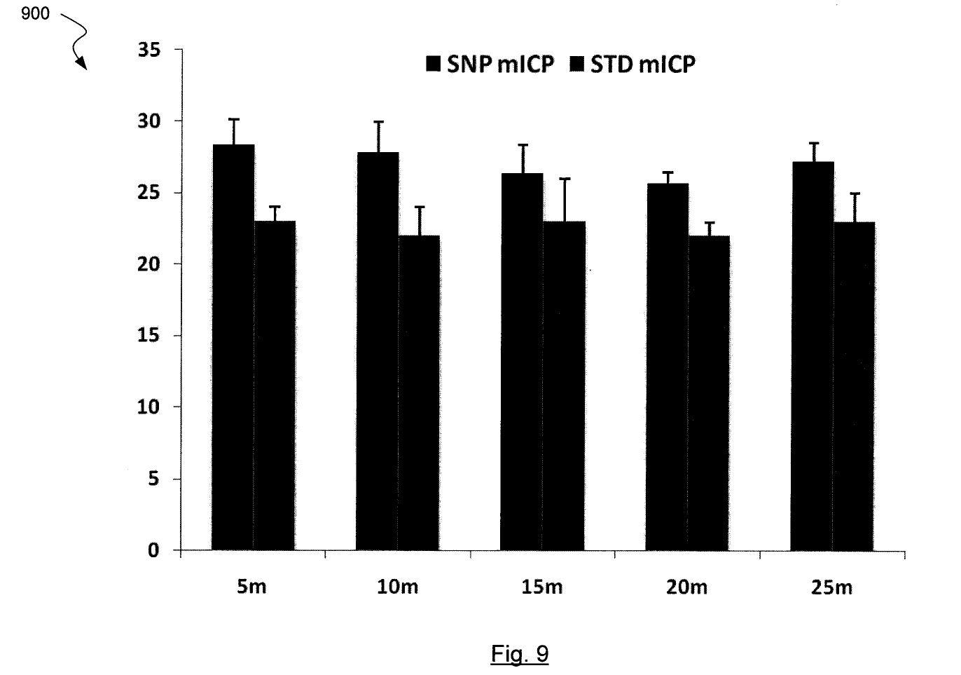

FIG. 9 shows a graph 900 of experimental results of mean intracranial pressure (mICP) over time during both standard CPR and SNP enhanced ACD CPR (including ITD and abdominal binding). The mICP produced by the SNP enhanced CPR (SNP mICP) is statistically significantly increased over the mICP produced by standard CPR (STD mICP) with a p-value of less than 0.05. Though statistically significant, the increase in mICP via SNP enhanced CPR is small, especially in light of the benefits of higher CPP and cerebral perfusion pressure (CerPP) (discussed below).

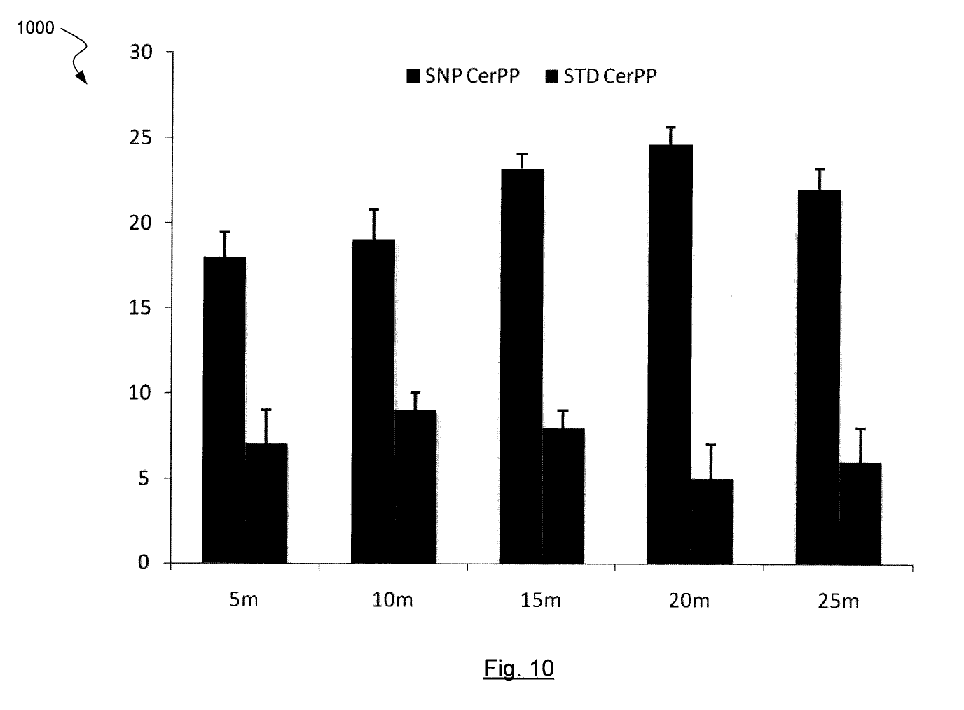

FIG. 10 shows a graph 1000 of experimental results of CerPP over time during both standard CPR and SNP enhanced ACD CPR (including ITD and abdominal binding). The CerPP produced by the SNP enhanced CPR (SNP CerPP) is statistically significantly increased over the CerPP produced by standard CPR (STD CerPP) with a p-value of less than 0.05. Improved CerPP using SNP enhanced ACD CPR over standard CPR results in greater blood flow to the brain, decreasing the likelihood and amount of neurological damage during CPR.

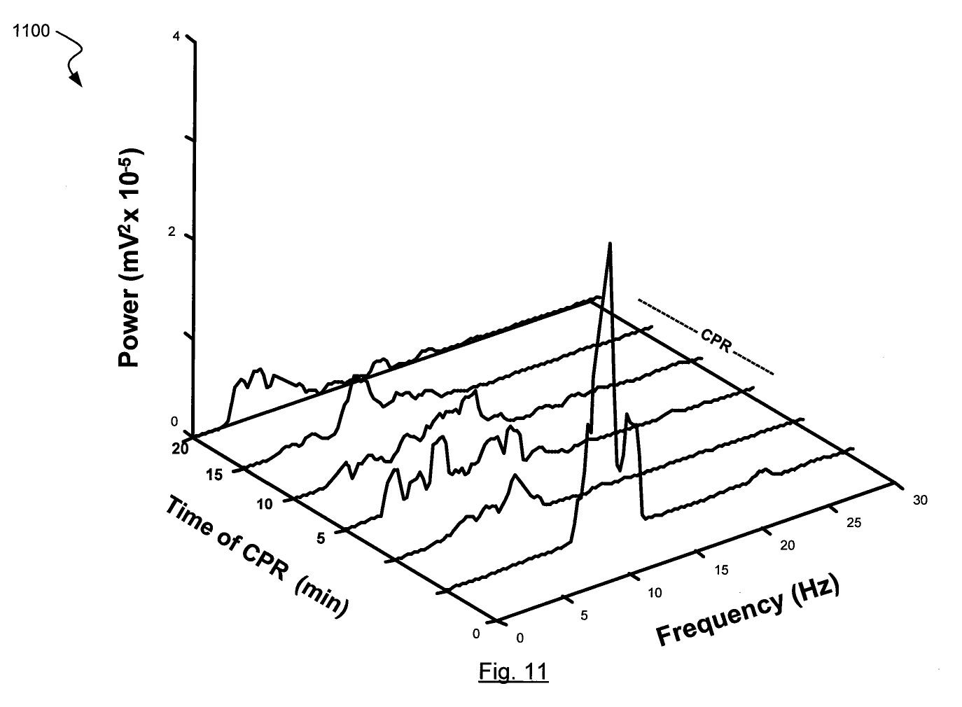

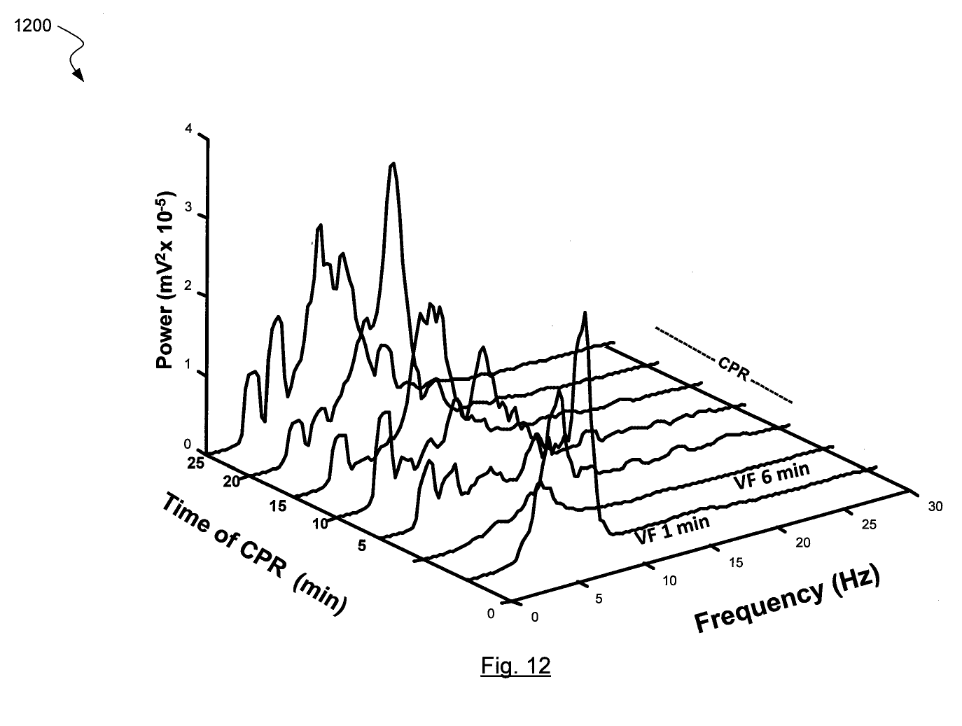

FIG. 11 shows a graph 1100 of a Fast Fourier Transformation (FFT) analysis of experimental results of ventricular fibrillation (VF) frequency and power over time during standard CPR. FIG. 12 shows a graph 1200 of a FFT analysis of experimental results of VF frequency and power over time during SNP enhanced ACD CPR (including ITD and abdominal binding). Improved VF frequency and power using SNP enhanced ACD CPR over standard CPR results in an increased likelihood that the subject will present a shockable rhythm that may result in ROSC when administered. In addition, the large high frequency signals observed with SNP, ACD CPR, ITD, and abdominal binding treatment can be used as a predictor for when the treatment efforts are likely to result in a successful resuscitation and/or when to administer more drug or electrical shock therapy.

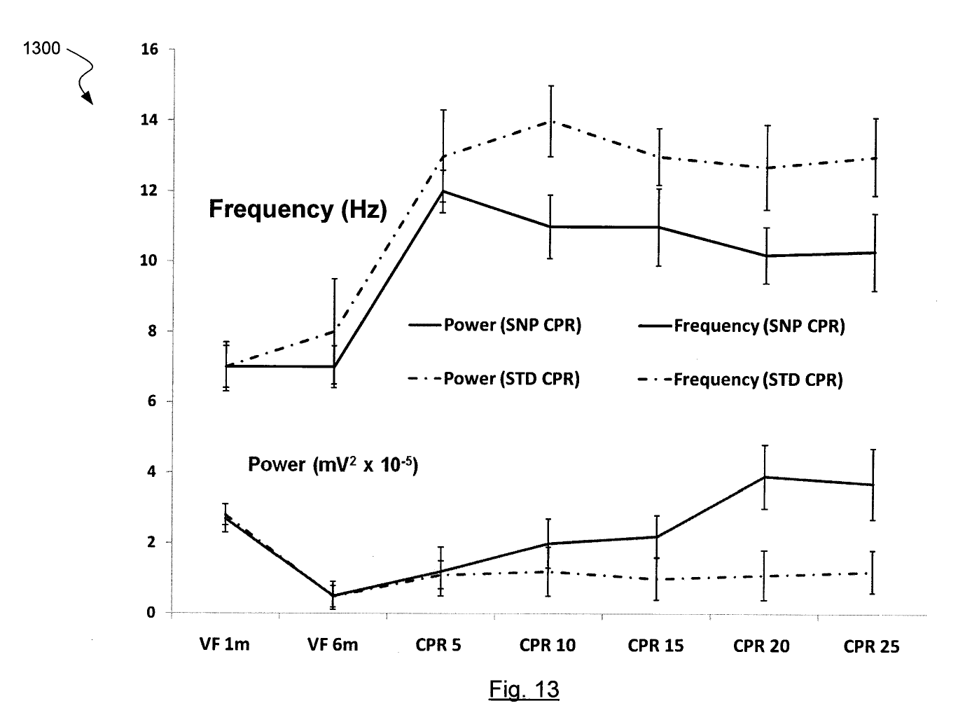

FIG. 13 shows a graph 1300 of experimental results of mean ventricular fibrillation frequency and power over time during both standard CPR and SNP enhanced ACD CPR (including ITD and abdominal binding). VF frequency and power are statistically significantly improved when using SNP enhanced ACD CPR over standard CPR (STD CPR) starting at least 10 minutes into resuscitation, with a p-value of less than 0.05. As discussed above, improved VF frequency and power using SNP enhanced ACD CPR over standard CPR results in an increased likelihood that the subject will present a shockable rhythm that may result in ROSC when administered.

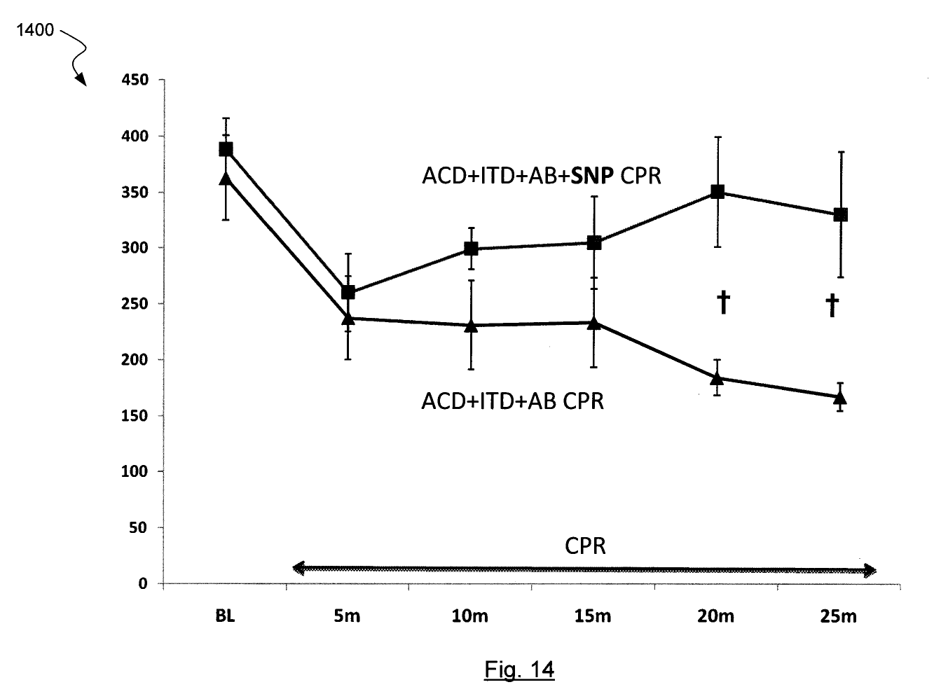

FIG. 14 shows a graph 1400 of experimental results of carotid blood flow (CBF) (in milliliters per minute) over time during both ACD CPR (including ITD and abdominal binding) and SNP enhanced ACD CPR (including ITD and abdominal binding). Beginning at 20 minutes, CBF produced by the SNP enhanced CPR is statistically significantly increased over the CBF produced the non-SNP assisted CPR with a p-value of less than 0.05. Improved CBF using SNP enhanced ACD CPR over standard CPR results in greater blood flow to the head and brain, decreasing the likelihood and amount of neurological damage during CPR.

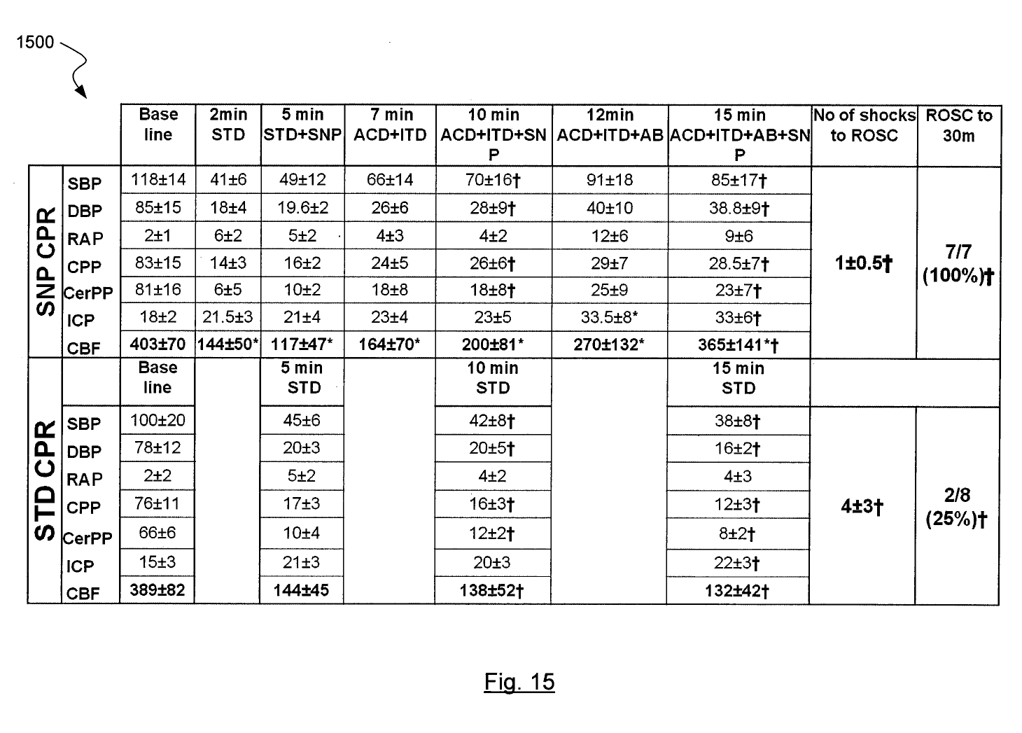

FIG. 15 shows a table 1500 of experimental results of hemodynamic parameters and the return of spontaneous circulation (ROSC) of both standard CPR and SNP enhanced CPR (including ITD and abdominal binding). The control group (eight pigs) received standard CPR only (bottom half of chart). The intervention group (also eight pigs) received standard CPR upon initiation of CPR, with SNP administered at 5 minutes. An ITD was employed at 7 minutes. SNP was again administered at 10 minutes, and an abdominal binding (AB) was employed at 12 minutes. At 15 minutes another administration of SNP occurred. The ITD used in these studies prevented respiratory gases from entering the lungs during the decompression phase of CPR. In these studies, the ITD had a safety check valve that would allow for inspiration when the intrathoracic pressure was less than minus 16 cm H2O, which does not occur during CPR. The abdomen was compressed with 40-50 lbs of pressure, applied continuously with a bend human arm, using the forearm and a force gauge.

Results with an “*” are statistically significantly different than other values for the same method (standard CPR or SNP enhanced CPR), with a p-value of less than 0.05. Results with a “ [symbol] ” are statistically significantly different than values for the other method (standard CPR versus SNP enhanced CPR), with a p-value of less than 0.05.

Table 1500 shows that the most significant differences between standard CPR and ACD CPR were for after prolonged periods of CPR when more and more methods ands systems (ACD, ITD, AB, and SNP) are employed. Notably, the number of defibrillatory shocks necessary to achieve ROSC is reduced, and ROSC is more likely, for SNP enhanced CPR.

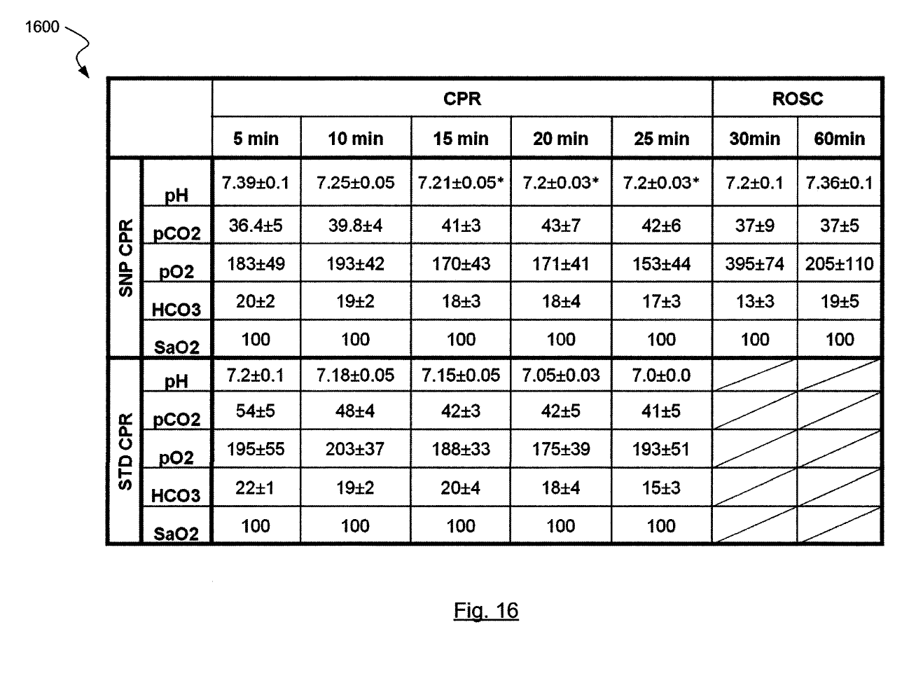

FIG. 16 shows a table 1600 of experimental results of basic arterial blood glasses of both standard CPR and SNP enhanced CPR (including ITD and abdominal binding). Results with an “*” are statistically significantly different than values for the other method (standard CPR versus SNP enhanced CPR), with a p-value of less than 0.05. pH approaches more normal levels for SNP enhanced CPR than standard CPR.

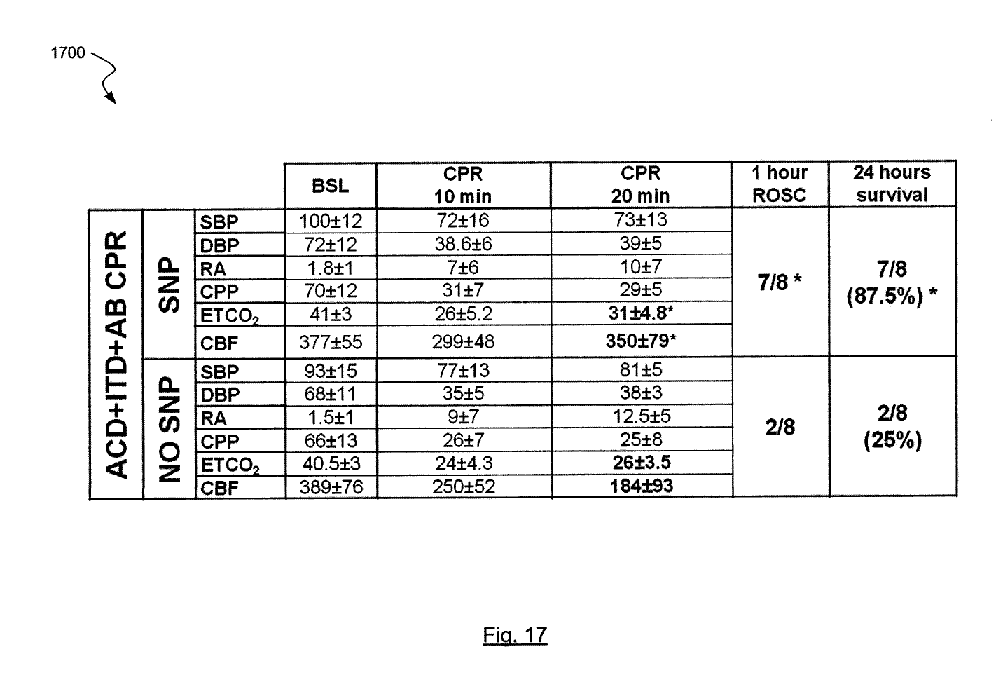

FIG. 17 shows a table 1700 of experimental results of hemodynamic and respiratory parameters of both non-SNP assisted ACD CPR (including ITD and abdominal binding) and SNP enhanced ACD CPR (including ITD and abdominal binding). Results with an “*” are statistically significantly different than values for the other method (non-SNP assisted ACD CPR versus SNP enhanced ACD CPR), with a p-value of less than 0.05.

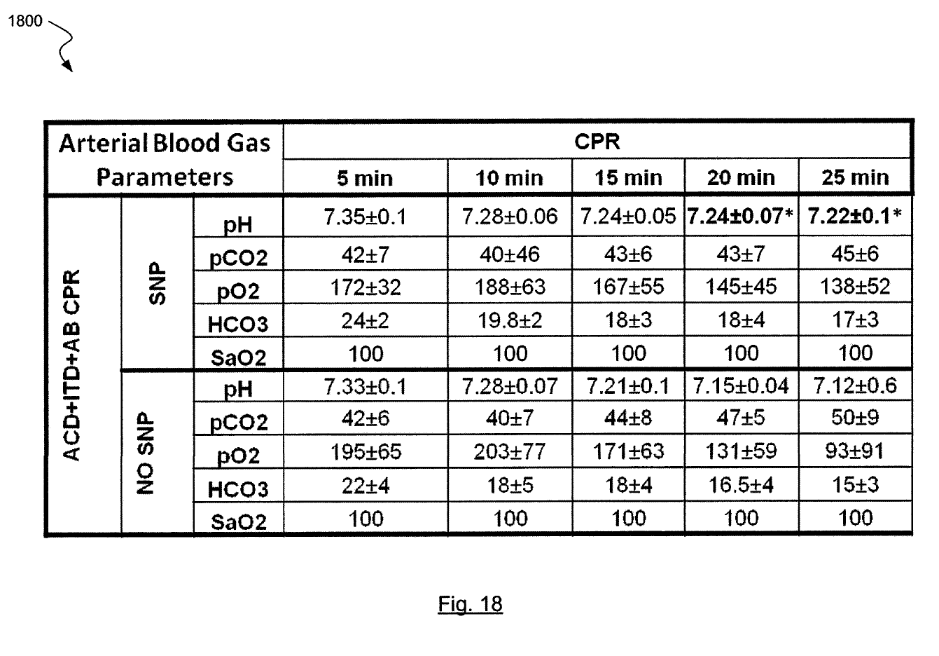

FIG. 18 shows a table 1800 of experimental results of arterial blood gas parameters of both non-SNP assisted ACD CPR (including ITD and abdominal binding) and SNP enhanced ACD CPR (including ITD and abdominal binding). Results with an “*” are statistically significantly different than values for the other method (non-SNP assisted ACD CPR versus SNP enhanced ACD CPR), with a p-value of less than 0.05.

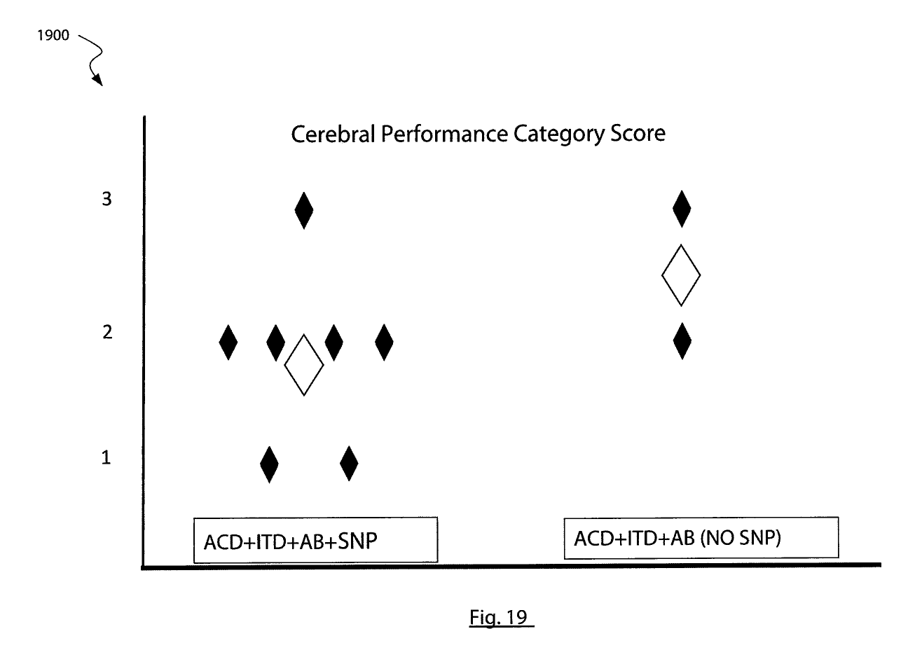

FIG. 19 shows a graph 1900 of experimental results of cerebral performance category scores of both non-SNP assisted ACD CPR (including ITD and abdominal binding) and SNP enhanced ACD CPR (including ITD and abdominal binding). “1” means normal cerebral performance, and “3” means severely disabled but conscious. Average values are shown by large diamonds, individual results by small diamonds. The SNP enhanced ACD CPR group shows better average cerebral performance than the non-SNP assisted ACD CPR group.

In addition to increasing circulatory to the heart and brain, SNP also causes vasodilatation of the blood supply to the skin. This promotes heat exchange, and in combination with the improved method of circulation (ACD CPR, ITD, ITPR, and/or abdominal binding) this approach can be used to facilitate and accelerate body cooling during and after cardiac arrest. Cooling, or therapeutic hypothermia, is a treatment to help preserve organ function, especially brain function. The novel embodiments described herein may enable clinicians to achieve the target hypothermic value faster and non-invasively, especially when used in conjunction with methods to cool the body, both non-invasively and invasively.

In addition to the systems and methods described above, embodiments of the invention include devices to aid in the delivery of the described therapies. Specifically, a device that binds the abdomen when SNP is being administered, such as a belt or pad upon which pressure can be applied, will help prevent blood from going into the lower abdomen and lower extremities, and instead encourage flow in the upper body. This binding could be administered at a given pressure (possibly in the range of about 20 pounds (lbs) to about 150 lbs), in a constant or pulsed manner. In some cases the pulsed abdominal pressure could be timed with the CPR cycle or application of the ITPR.

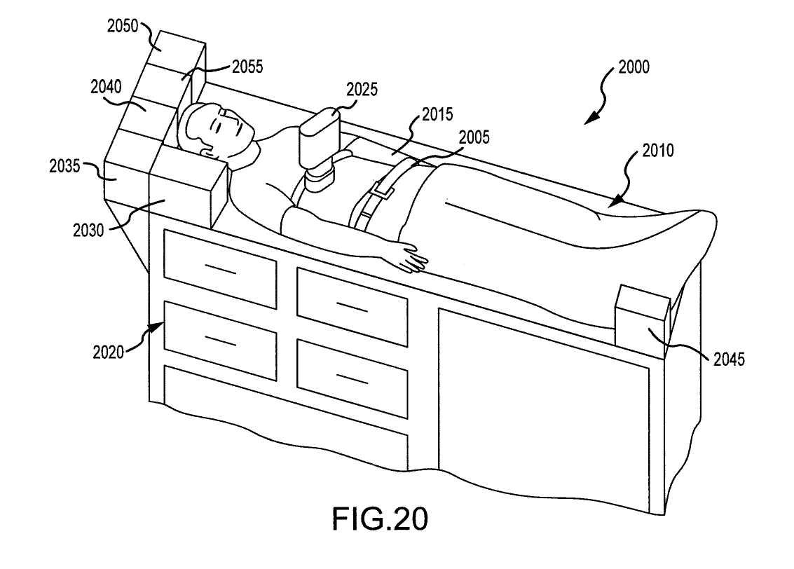

Building upon this abdominal binder, with or without a pressure gauge, the pressure could be adjusted based upon a physiological measurement, for example blood pressure or brain perfusion. Further, as shown in FIG. 20, in such an embodiment 2000 an abdominal binder 2005 could be attached to a back board 2010 to stabilize abdominal binder 2005 and the patient 2015. In some embodiments, abdominal binder 2005 could be narrower or wider than shown in FIG. 20. In yet other embodiments, the shape of the abdominal binder could be configured to conform, at least partially, to the lower torso and/or upper legs of the person.

Such an embodiment 2000 may be a workstation as shown in FIG. 20, and include storage compartments 2020 underneath back board 2010 (though some embodiments may exclude such compartments 2020). In some embodiments, the back board 2010 may simply be a flat board, or a flat board stretcher/gurney. In some embodiments, the back board may be made from collapsible and/or flexible subsections, possibly similar to a canvas stretcher used by military personnel. In yet other embodiments, backboard may be a collapsible gurney as commonly used in emergency service ambulances. Back board 2010 could be further extended to behind the head and thorax of patient 2015 as also shown in FIG. 20. Back board 2010 could have a cradle in it to house the CPR device chest compression device 2025, such as the bottom part of the LUCAS device. Back board 2010 could also include equipment necessary to deliver SNP via intravenous or other methods.

Backboard 2010 could serve as a CPR workstation, with space for a defibrillator 2030, an ITPR 2035 (automated or mechanical), a cooling device 2040, and/or a lower extremity counterpulsation device 2045. In addition, back board 2010 may have a control station 2050 to regulate chest compressions, ITPR, cooling, abdominal binding/counterpulsation, and defibrillation, all possibly with an electronic patient management system complete with a drug infusion pump for administration of drugs to the patient through an IV or intra-osseous delivery mechanism. Consequently, via control station 2050, any of the above devices could collect data from one another and/or control/direct the actions of another. Merely by way of example, a blood pressure and/or EKG monitor 2055 could monitor the blood pressure and hear activity of person 2015, and direct the functioning of other devices on the back board 2010. In yet another example, abdominal binding 2005 could be dynamic, and adjust periodically or continually based on parameters of person 2015 as determined by other devices on back board 2010.

The invention has now been described in detail for the purposes of clarity and understanding. However, it will be appreciated that certain changes and modifications may be practiced within the scope of the appended claims.

What is claimed is:

1. A kit for increasing blood flow to vital organs during cardiopulmonary resuscitation of a person experiencing a cardiac arrest, wherein the kit comprises:
one or more vasodilator drugs in an amount effective to improve artificial circulation during cardiopulmonary resuscitation when administered to the person; and
instructions for performing cardiopulmonary resuscitation and for administering the one or more vasodilator drugs in the effective amount.

2. The kit for increasing blood flow to vital organs during cardiopulmonary resuscitation of claim 1, wherein one or more vasodilator agents is selected from a group consisting of:
sodium nitroprusside;
glycerol trinitrate;
isosorbide mononitrate;
isosorbide dinitrate;
pentaerythritol tetranitrate;
sildenafil;
tadalafil;
vardenafil.
adenosine
an adenosine analog and
a methylxanthine.

3. The kit for increasing blood flow to vital organs during cardiopulmonary resuscitation of claim 1, further comprising a mechanical device to increase the blood pressure of the person, wherein the mechanical device is selected from a group consisting of:
an abdominal binding;
an impedance threshold device;
an intrathoracic pressure regulator;
an automated chest compression device; and
an active compression decompression chest compression device.

4. The kit for increasing blood flow to vital organs during cardiopulmonary resuscitation of claim 3, wherein the kit further comprises a support surface, wherein:
the support surface is configured to support the person experiencing the cardiac arrest; and
the support surface is coupled with the mechanical device.

5. The kit for increasing blood flow to vital organs during cardiopulmonary resuscitation of claim 1, wherein the kit further comprises at least one device to assist in inducing therapeutic hypothermia in the person.

6. The kit for increasing blood flow to vital organs during cardiopulmonary resuscitation of claim 5, wherein inducing therapeutic hypothermia in the person comprises lowering the person's body temperature to between about 32° C. and about 34° C.

7. A method for increasing blood flow to vital organs during cardiopulmonary resuscitation of a person experiencing a cardiac arrest, wherein the method comprises:
administering one or more vasodilator agents to the person; and
mechanically increasing blood pressure in addition to providing cardiopulmonary resuscitation to the person.

8. The method for increasing blood flow to vital organs during cardiopulmonary resuscitation of claim 7, wherein the method further comprises inducing therapeutic hypothermia in the person.

9. A method for increasing blood flow to vital organs during cardiopulmonary resuscitation of a person experiencing a cardiac arrest, wherein the method comprises:
performing cardiopulmonary resuscitation on a person to create artificial circulation by repetitively compressing the person's chest such that the person's chest is subject to a compression phase and a relaxation or decompression phase; and
administering sodium nitroprusside by itself or in combination with adenosine to the person to improve the artificial circulation created by the cardiopulmonary resuscitation.

10. A method for increasing blood flow to vital organs during cardiopulmonary resuscitation of a person experiencing a cardiac arrest, wherein the method comprises:
performing cardiopulmonary resuscitation on a person to create artificial circulation by repetitively compressing the person's chest such that the person's chest is subject to a compression phase and a relaxation or decompression phase; and
administering sodium nitroprusside by itself or in combination with adenosine in part of a cocktail of compounds designed to preserve brain function, to the person to improve the artificial circulation created by the cardiopulmonary resuscitation.

11. The method for increasing blood flow to vital organs during cardiopulmonary resuscitation of claim 10, wherein performing cardiopulmonary resuscitation comprises:
performing standard cardiopulmonary resuscitation; or
performing active compression decompression cardiopulmonary resuscitation.

12. The method for increasing blood flow to vital organs during cardiopulmonary resuscitation of claim 10, wherein the method further comprises:
binding at least a portion of the person's abdomen.

13. The method for increasing blood flow to vital organs during cardiopulmonary resuscitation of claim 12, wherein the binding at least a portion of the person's abdomen comprises:
binding, manually, at least a portion of the person's abdomen; or
binding, with an abdominal compression device, at least a portion of the person's abdomen.

14. The method for increasing blood flow to vital organs during cardiopulmonary resuscitation of claim 10, wherein the method further comprises:
binding, manually, at least a portion of the person's abdomen; or
binding, with an abdominal compression device, at least a portion of the person's abdomen.

15. The method for increasing blood flow to vital organs during cardiopulmonary resuscitation of claim 12, further comprising:
at least temporarily preventing or impeding airflow to the person's lungs during at least a portion of the relaxation or decompression phase using an impedance threshold device that is coupled with the person's airway.

16. The method for increasing blood flow to vital organs during cardiopulmonary resuscitation of claim 11, further comprising:
at least temporarily preventing or impeding airflow to the person's lungs during at least a portion of the relaxation or decompression phase using an impedance threshold device that is coupled with the person's airway.

17. The method for increasing blood flow to vital organs during cardiopulmonary resuscitation of claim 10, further comprising:
at least temporarily preventing or impeding airflow to the person's lungs during at least a portion of the relaxation or decompression phase using an impedance threshold device that is coupled with the person's airway.

18. The method for increasing blood flow to vital organs during cardiopulmonary resuscitation of claim 12, further comprising:
regulating the airflow to or from the person's lungs using an intrathoracic pressure regulator.

19. The method for increasing blood flow to vital organs during cardiopulmonary resuscitation of claim 10, further comprising:
regulating the airflow to or from the person's lungs using an intrathoracic pressure regulator.

20. The method for increasing blood flow to vital organs during cardiopulmonary resuscitation of claim 10, further comprising measuring a blood pressure of the person, and further:
altering the administration of sodium nitroprusside based on the blood pressure; or
altering a manner of chest compressions based on the blood pressure.

21. The method for increasing blood flow to vital organs during cardiopulmonary resuscitation of claim 10, further comprising:
ventilating the person, at least periodically.

22. The method for increasing blood flow to vital organs during cardiopulmonary resuscitation of claim 21, further comprising:
measuring a blood pressure of the person; and
altering the ventilating of the person based on the blood pressure.

23. The method for increasing blood flow to vital organs during cardiopulmonary resuscitation of claim 10, wherein the method further comprises inducing hypothermia in the person.

24. The method for increasing blood flow to vital organs during cardiopulmonary resuscitation of claim 10, wherein the method further comprises delivery of a defibrillatory shock to the person.

25. The method for increasing blood flow to vital organs during cardiopulmonary resuscitation of claim 10, wherein administering sodium nitroprusside to the person comprises:
administering sodium nitroprusside in amounts of at least substantially 1 milligram per 30 kilograms of mass of the person (1 milligram per 66 pounds of weight of the person).

26. The method for increasing blood flow to vital organs during cardiopulmonary resuscitation of claim 10, wherein administering sodium nitroprusside to the person comprises:
administering at least two doses of sodium nitroprusside with at least a 3 to 5 minute interval between at least two doses.

27. The method for increasing blood flow to vital organs during cardiopulmonary resuscitation of claim 10, wherein improving the artificial circulation created by the cardiopulmonary resuscitation comprises:
increasing carotid blood flow.

28. The method for increasing blood flow to vital organs during cardiopulmonary resuscitation of claim 10, wherein improving the artificial circulation created by the cardiopulmonary resuscitation comprises:
increasing systolic and diastolic blood pressures.

29. The method for increasing blood flow to vital organs during cardiopulmonary resuscitation of claim 10, wherein administering sodium nitroprusside further improves a favorable characteristic of a ventricular fibrillation waveform of the person at a point in time after an onset of the cardiac arrest.

30. The method for increasing blood flow to vital organs during cardiopulmonary resuscitation of claim 10, wherein the method further comprises inducing therapeutic hypothermia in the person.

31. The method for increasing blood flow to vital organs during cardiopulmonary resuscitation of claim 12, wherein the method further comprises inducing therapeutic hypothermia in the person.

32. The method for increasing blood flow to vital organs during cardiopulmonary resuscitation of claim 15, wherein the method further comprises inducing therapeutic hypothermia in the person.

33. The method for increasing blood flow to vital organs during cardiopulmonary resuscitation of claim 17, wherein the method further comprises inducing therapeutic hypothermia in the person.

34. The method for increasing blood flow to vital organs during cardiopulmonary resuscitation of claim 18, wherein the method further comprises inducing therapeutic hypothermia in the person.

35. The method for increasing blood flow to vital organs during cardiopulmonary resuscitation of claim 19, wherein the method further comprises inducing therapeutic hypothermia in the person.

36. A method for increasing blood flow to vital organs during cardiopulmonary resuscitation using an at least partially invasive circulatory assist procedure, wherein the method comprises:
performing cardiopulmonary resuscitation on a person by repetitively compressing the person's chest such that the person's chest is subject to a compression phase and a relaxation or decompression phase;
performing an at least partially invasive circulatory assist procedure on the person; and
administering sodium nitroprusside to the person.

37. The method for increasing blood flow to vital organs during cardiopulmonary resuscitation using an at least partially invasive circulatory assist procedure of claim 36, wherein the at least partially invasive circulatory assist procedure comprises:
inserting a percutaneous left ventricular assist device into the person;
inserting an intra-aortic balloon pump into the person; or
performing a cardiopulmonary bypass on the person.

38. A method for increasing blood flow to vital organs of a person experiencing a cardiac arrest, wherein the method comprises:
alternatively compressing and decompressing a chest of the person at a rate of about 60 cycles to about 120 cycles per minute to create artificial circulation;
administering sodium nitroprusside in an amount of about 0.01 milligrams to about 5.0 milligrams to the person to improve the artificial circulation created by the alternative compressing and lifting of the chest;
regulating inflow of respiratory gases into the person's lungs during decompressing of the chest to maintain a negative intrathoracic pressure at least below about −4.0 millimeters of Mercury (mmHg) for a time of at least about 1.0 seconds.

39. The method for increasing blood flow to vital organs of a person experiencing a cardiac arrest of claim 38, wherein the method further comprises:
binding at least a portion of the person's abdomen.

40. The method for increasing blood flow to vital organs of a person experiencing a cardiac arrest of claim 38, wherein regulating inflow of respiratory gases into the person's lungs during decompressing of the chest comprises:
disposing a threshold valve in communication with the person's airway, wherein the threshold valve is set to open in a range from about −2.0 centimeters water (cmH20) to about −20 cmH20.

41. The method for increasing blood flow to vital organs of a person experiencing a cardiac arrest of claim 38, wherein regulating inflow of respiratory gases into the person's lungs during decompressing of the chest comprises:
extracting gases from the lungs using a vacuum source.

42. The method for increasing blood flow to vital organs of a person experiencing a cardiac arrest of claim 41, wherein the person's lungs experience a vacuum having a pressure of less than about −4.0 millimeters of mercury (mmHg).

43. The method for increasing blood flow to vital organs of a person experiencing a cardiac arrest of claim 41, wherein:
a start time of the vacuum is substantially coincident with a start of the decompressing of the chest; and
an end time of the vacuum is substantially coincident with an end of the compression of the chest.

44. The method for increasing blood flow to vital organs of a person experiencing a cardiac arrest of claim 38, further comprising measuring a blood pressure of the person, and further:
altering the administration of sodium nitroprusside based on the blood pressure; or
altering a manner of chest compressions based on the blood pressure.

45. A method of inducing therapeutic hypothermia in a person, wherein the method comprises:
administering a vasodilator to the person; and
lowering the temperature of the person.

46. The method of inducing therapeutic hypothermia in a person of claim 45, wherein administering the vasodilator to the person comprises administering sodium nitroprusside to the person.

47. The method of inducing therapeutic hypothermia in a person of claim 45, wherein lowering the temperature of the person comprises lowering the temperature of the heart or the brain of the person to between about 32° C. and about 34° C.
